(19) United States
(12) Patent Application Publication
(10) Pub. No.: US 2026/0176656 A1
(43) Pub. Date: Jun. 25, 2026

(54) HIGH PROTEIN YEAST PRODUCT

(71) Applicant: DANSTAR FERMENT AG, Zug (CH)

(72) Inventors: Stanislaw PTASZYNSKI, Toronto (CA); Jan-Martijn HAMELINK, Toronto (CA); Annemarie HALTER, Zug (CH)

(73) Assignee: DANSTAR FERMENT AG, Zug (CH)

(21) Appl. No.: 19/125,161

(22) PCT Filed: Oct. 26, 2023
(86) PCT No.: PCT/IB2023/060817

§ 371 Date: Apr. 28, 2025

Related U.S. Application Data
(60) Provisional application No. 63/381,353, filed on Oct. 28, 2022.

(51) Int. Cl. C12P 7/16; A23K 10/38; A61K 31/202; A61K 38/00; A61K 39/12

(52) U.S. Cl. CPC C12P 7/16; A23K 10/38; A61K 31/202; A61K 38/00; A61K 39/12; A23V 2002/00

(57) ABSTRACT

The present disclosure concerns a method of obtaining a composition having yeast proteins derived from yeast protoplasts. The present disclosure also includes compositions comprising yeast proteins derived from yeast protoplasts, edible products derived from said composition as well as usages thereof.

### CROSS-REFERENCE TO RELATED APPLICATION(S) AND DOCUMENT(S)

This is patent application claims priority from U.S. provisional patent application 63/381,353 filed on Oct. 28, 2023 and herewith incorporated in its entirety.

### TECHNOLOGICAL FIELD

The present disclosure concerns yeast protein having neutral taste, composition thereof and method of producing the same.

### BACKGROUND

Proteins are biological macromolecules that are essential components of animal nutrition. Once ingested, proteins are degraded by proteases into polypeptides, then into amino acids. The latter will then be able to reach the bloodstream by crossing the intestinal barrier, and then spread throughout the body in order to assume their vital functions.

In human and/or animal nutrition, the main protein sources are animal proteins, plant proteins (including leguminous proteins) and fungal proteins. The consumption of animal proteins has been growing steadily for the last 50 years and therefore raises environmental issues as well as health concerns for consumers. Thus, the current nutritional challenge is to reverse the trend in terms of protein consumption in the world, namely: reduce the share of animal proteins and increase those of plant/leguminous proteins (including pea proteins for example) and fungal proteins such as those found in a yeast extract.

However, proteins from yeast extracts as well as plant/leguminous proteins have a distinctive taste which limits their concentration and therefore their applications. The disclosure herein is seeking to provide a source of proteins having a neutral taste to address these issues.

### SUMMARY

According to a first aspect, the present disclosure provides a method for obtaining a composition having yeast proteins. Broadly, the method comprises: a) providing a yeast cream comprising yeasts; b) inactivating the endogenous enzymes of the yeasts to provide an inactivated yeast cream; c) subjecting the inactivated yeast cream to an enzymatic treatment to obtain an insoluble fraction comprising yeast protoplasts and a soluble fraction; d) separating the insoluble fraction from the soluble fraction; and e) collecting the insoluble fraction, wherein the collected insoluble fraction, when dried, is the composition comprising the yeast proteins and has a protein content equal to or higher than 60% based on the total mass of the dried collected insoluble fraction. At step c), the enzymatic treatment comprises at least one polypeptide having a glucanase activity; and the enzymatic treatment lacks ribonuclease activity. In one embodiment, the inactivated yeast cream is obtained by exposing the yeast cream to a temperature of between 65 and 100° C., for a time of between 30 seconds and 5 hours. In an embodiment, the enzymatic treatment is performed at a temperature of between 2° and 80° C., for a time of between 1 and 24 hours. In another embodiment, the composition has a neutral taste. In some embodiments, the method further comprising drying the collected insoluble fraction of step e) to provide the composition. In some another embodiment, the dried insoluble fraction has a lipid content lower than 20% based on the total mass of the dried collected insoluble fraction; a nucleic acid content higher than 6% based on the total mass of the dried collected insoluble fraction; a carbohydrate content lower than 25% based on the total mass of the dried collected insoluble fraction; a mannan content lower than 6% based on the total mass of the dried collected insoluble fraction; a glucan content lower than 10% based on the total mass of the dried collected insoluble fraction; and/or a glucose content lower than 25% based on the total mass of the dried collected insoluble fraction. In some embodiments, the method can further comprise, after b) and before c), submitting the inactivated yeast cream to an alkaline extraction step. In some further embodiment, the method can further comprise drying the collected insoluble fraction of step e) (which has been submitted to an alkaline extraction step) to provide the composition. In such embodiments, the dried insoluble fraction can have a lipid content lower than 20% based on the total mass of the dried collected insoluble fraction; a nucleic acid content lower than 3% based on the total mass of the dried collected insoluble fraction; a carbohydrate content lower than 25% based on the total mass of the dried collected insoluble fraction; a mannan content lower than 6% based on the total mass of the dried collected insoluble fraction; a glucan content lower than 10% based on the total mass of the dried collected insoluble fraction; and/or a glucose content lower than 25% based on the total mass of the dried collected insoluble fraction. In a further embodiment, the yeasts are from the genuses Saccharomyces, Komagataella, Pichia, Candida, Kluyveromyces, Yarrowia, Cyberlindera (Torula), Wickerhamomyces, or a combination thereof.

According to a second aspect, the present disclosure provides a composition comprising yeast proteins being derived from yeast protoplasts and having a protein content equal to or higher than 60% based on the total mass of the composition; a lipid content lower than 20% based on the total mass of the composition; a carbohydrate content lower than 25% based on the total mass of the composition; a mannan content lower than 6% based on the total mass of the composition; a glucan content lower than 10% based on the total mass of the composition; and/or a glucose content lower than 25% based on the total mass of the composition. In some embodiments, the composition can have a nucleic acid content higher than 6% based on the total mass of the composition. In other embodiments, the composition can have a nucleic acid content lower than 3% based on the total mass of the composition. In one embodiment, the composition having a neutral taste. In an embodiment, the composition is obtainable or obtained by the method for obtaining a composition having yeast proteins.

According to a third aspect, the present disclosure provides an edible product comprising the composition and at least one further ingredient, wherein the composition provides at least 1% w/w, based on the total mass of edible product. In an embodiment, the edible product is a beverage, a shake, a bar, a meat product or a baked product.

According to a fourth aspect, the present disclosure provides the use of the edible product in human and/or animal nutrition. In one embodiment, the edible product provides as an alternative to animal or plant/leguminous proteins-based edible products, or is combined with animal or plant/leguminous proteins-based edible products. In an embodiment, the edible product is a food/feed supplement or food/feed additive. In another embodiment, the food/feed supplement or the food/feed additive is for weight control, for elderly, for oral/enteral clinical nutrition, for sports applications and/or for animal nutrition.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 shows an embodiment of the method for making compositions comprising yeast proteins (composition C), as well as comparative products (compositions A and B).

FIGS. 2A1, 2A2, FIGS. 2B1, 2B2, FIGS. 2C1 and 2C2 show the organoleptic properties (i.e., smell and taste) based on the Rate-All-That-Apply (RATA) method performed on Composition A (FIGS. 2A1 and 2A2), Composition B (FIGS. 2B1 and 2B2) and on Nutralys® F85M (FIGS. 2C1 and 2C2). * refers to mandatory fields requested for the organoleptic evaluation of the compositions.

FIGS. 3A to 3E show the Texture Profile Analysis (TPA) of extrudates (i.e., H0, H5, H10, H15, H25 and H35). Results are shown for (A) chewiness, (B) cohesion, (C) hardness, (D) resilience and (E) springiness. Vertical error bars show 95% confidence intervals, and samples not sharing a letter are statistically significantly different (Kruskal-Wallis test, p<0.05). H100 was too strong and was not measured.

FIGS. 4A to 4I show the Descriptive Sensory Analysis (DSA) of extrudates (i.e., H0, H5, H10, H15, H25, H35 and H100) which evaluated odor (O) and taste (T) attributes. Results are shown for the odor of (A) legumes, (B) meaty and yeasty, (C) off-odor and (D) overall intensity as well as the (E) after-taste intensity, the taste of (F) legumes, (G) off-flavor intensity, (H) overall intensity as well as (I) umami. Vertical error bars show 95% confidence intervals, and samples not sharing a letter are statistically significantly different (Kruskal-Wallis test, p<0.05).

FIGS. 5A to 5H show the Descriptive Sensory Analysis (DSA) of extrudates (i.e., H0, H5, H10, H15, H25, H35 and H100) which evaluated texture attributes (X). Results are shown as the attributes of (A) adhesiveness, (B) chewiness, (C) coherence, (D) fibrousness, (E) granularity, (F) hardness, (G) moistness, and (H) springiness. Vertical error bars show 95% confidence intervals, and samples not sharing a letter are statistically significantly different (Kruskal-Wallis test, p<0.05).

### DETAILED DESCRIPTION

### Method for Obtaining a Composition Having Yeast Proteins

In one embodiment, the present disclosure aims at providing a method for obtaining a composition having yeast proteins. The method of the present disclosure comprises:

a) providing a yeast cream comprising yeasts; b) inactivating the endogenous enzymes of the yeasts to provide an inactivated yeast cream; c) subjecting the inactivated yeast cream to an enzymatic treatment to obtain an insoluble fraction comprising yeast protoplasts and a soluble fraction; d) separating the insoluble fraction from the soluble fraction; and e) collecting the insoluble fraction.

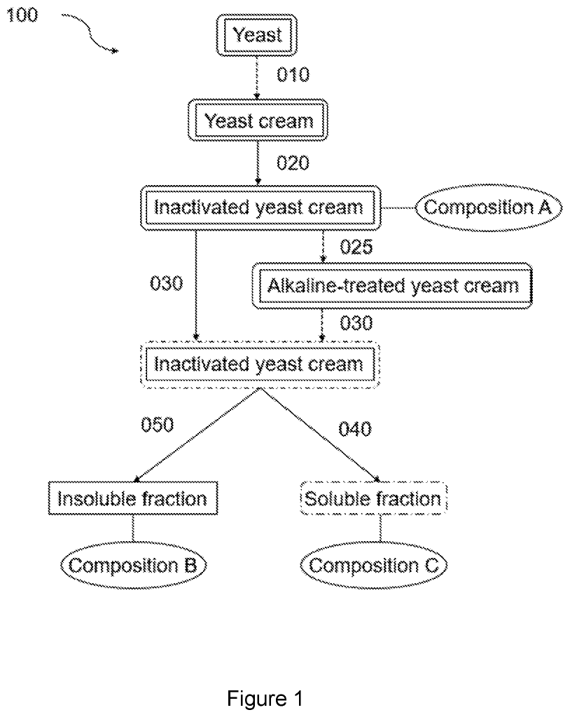

In the context of the present disclosure, the term “yeast” refers to eukaryotic, single-celled microorganisms belonging to the fungi kingdom. Suitable yeasts that can be used in the method to obtain the composition can be, for example, from the genus Saccharomyces, Komagataella, Pichia, Candida, Kluyveromyces, Yarrowia, Cyberlindnera (Torula), Wickerhamomyces, or a combination thereof. Suitable yeast species that can be used in the method to obtain de composition can include, without being limited to, for example, Saccharomyces cerevisiae, Cyberlindnera jadinii, Komagataella phaffii (Pichia pastoris), Yarrowia lipolytica, Candida glabrata, Kluyveromyces lactis, Kluyveromyces marxianus, Wickerhamomyces anomalus, Debaryomyces hansenii or a combination thereof. In some embodiments, the yeast species is selected from the group consisting of Saccharomyces cerevisiae, Cyberlindnera jadinii, or a combination thereof. In an embodiment, the yeast is from the genus Saccharomyces and, in some embodiments, from the species Saccharomyces cerevisiae. In an embodiment, the yeast is from the genus Cyberlindnera and, in some embodiments, from the species Cyberlindnera jadinii. An embodiment of a method for obtaining a composition comprising yeast proteins is provided in FIG. 1. In the method 100 of FIG. 1, a yeast cream is provided. In an embodiment, the method can optionally include propagating yeasts (not shown on FIG. 1). The yeast propagation is performed by the one with ordinary skills in the art, respecting the appropriate methods known in the art. As used in the context of the present disclosure, the expression “yeast propagation” refers to an expansion phase of a commercial method in which the yeasts are propagated under aerobic conditions to maximize the conversion of a substrate into biomass. The propagation step can be a continuous method, a batch method or a fed-batch method. The propagation medium can comprise a carbon source (such as, for example, molasses, sucrose, glucose, dextrose syrup, ethanol, corn, glycerol, corn steep liquor and/or a lignocellulosic biomass), a nitrogen source (such as, for example, ammonia or another inorganic source of nitrogen) and a phosphorous source (such as, for example, phosphoric acid or another inorganic source of phosphorous). The propagation medium can further comprise additional micronutrients such as vitamins and/or minerals to support the propagation of the yeast cell.

The propagation process can be conducted under high aeration conditions. For example, in some embodiments, the propagation step can include controlling the aeration of the vessel to achieve a specific aeration rate, for example, of at least 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 air volume/vessel volume/minute.

The propagation step can be conducted at a specific pH and/or a specific temperature which is optimal for yeast biomass production. As such, in embodiments in which the yeast is from the genus Saccharomyces or Cyberlindnera, the process can comprise controlling the pH of the culture medium to between about 3.0 to about 8.0, about 3.5 to about 7.0 or about 4.0 to about 6.5. In a specific embodiment, the pH is controlled at about 4.5. In another example, in embodiments in which the yeast is from the genus Saccharomyces or Cyberlindnera, the process can comprise controlling the temperature of the culture medium between about 20° C. to about 40° C., about 25° C. to about 30° C. or about 30° C. to about 35° C. In a specific embodiment, the temperature is controlled at between about 30° C. to about 35° C. (32° C. for example).

At the end of the propagation step, a specific concentration can be sought or achieved. In some embodiments, the concentration of the propagated yeast cell in the culture medium is at least about 0.1, 0.15, 0.2, 0.25, 0.3, 0.35, 0.4, 0.45, 0.5, 1.0, 1.5, 2.0, 2.5, 3.0, 3.5, 4.0, 4.5, 5.0 or more weight % with respect to the volume of the culture medium. In a specific embodiment in which the yeast cell is propagated using a fed-batch process, the concentration of the propagated yeast cell in the culture medium is at least about 0.25 weight % with respect to the volume of the culture medium.

In some embodiments, the propagated yeasts are directly submitted to a formulation step to provide a yeast cream. For example, the propagated yeasts are not submitted to a following fermentation step (which may be conducted in anaerobic conditions) prior to the formulation step.

In the formulating step 010, the mixture obtained after propagation (comprising the propagated yeast cell(s)) is modified to provide a yeast cream. For example, at least one component of the mixture obtained after propagation is removed from the culture medium to provide a yeast composition (a yeast cream, an embodiment of a yeast composition, is provided as an embodiment on FIG. 1). This at least one component can be, without limitation, water, amino acids, peptides and proteins, nucleic acid residues and nucleic acid molecules, cellular debris, fermentation products, etc. In an embodiment, the formulating step 010 comprises substantially isolating the propagated yeast cells (e.g., the biomass) from the components of the culture medium. As used in the context of the present disclosure, the expression “substantially isolating” refers to the removal of the majority of the components of the culture medium from the propagated yeast cells. In some embodiments, “substantially isolating” refers to concentrating the propagated yeast cell to at least 5, 10, 15, 20, 25, 30, 35, 45, 50, 60, 70, 80, 90, 100, 110, 120, 130, 140, 150, 160, 170, 180, 190, 200, 210, 220, 230, 240, 250, 260, 270, 280, 290, 300% or more when compared to the concentration of the yeast cell within the culture medium prior to the isolation. In order to provide the yeast composition, the propagated yeast cells can be centrifuged (and the resulting cellular pellet comprising the propagated yeast cells can optionally be washed) and/or filtered. The isolated yeast cells can then be formulated in a yeast composition (which can be a yeast cream as provided on FIG. 1). The formulation step 010 can, in some embodiments, preserve the viability (at least in part) of the yeast cells. As such, the propagated yeasts can be provided in an active or a semi-active form. The propagated yeasts can be provided in a liquid or semi-solid form. In an embodiment, the propagated yeasts can be provided in the form of a yeast cream as shown on FIG. 1.

In the methods of the present disclosure, the endogenous enzymes of the yeast comprised in the yeast cream are inactivated to provide an inactivated yeast cream. This is shown as step 020 on FIG. 1. Such yeast endogenous enzymes inactivation step is performed to limit/avoid yeast autolysis. In the context of the present disclosure, the term “autolysis” represents self-degradation of the cellular constituents of a yeast cell by its own enzymes. Hence, the yeast endogenous enzymes inactivation step 020 is performed to limit/avoid degradation of cellular constituents (especially yeast proteins) which predominantly corresponds to the breakdown of proteinaceous substances (i.e., proteolysis). In one embodiment, the endogenous enzymes inactivation does not cause thermal plasmolysis. In the context of the present disclosure, the expression “thermal plasmolysis” refers to yeast denaturation and permeabilization of the yeast membrane. In another embodiment, the endogenous enzyme inactivation of the disclosure herein does not permeabilize the yeast membrane. In yet another embodiment, the endogenous enzyme inactivation keeps the yeast membrane integrity. In an embodiment, the endogenous enzymes inactivation is obtained by exposing the yeast cream to temperature and/or pH variations for a period of time. In one embodiment, the endogenous enzymes inactivation is obtained by exposing the yeast cream at a temperature higher than 4° C. and lower than 120° C. In an embodiment, the endogenous enzymes inactivation is obtained by exposing the yeast cream at a temperature of at least 5, 10, 15, 20, 25, 30, 35, 40, 45, 50, 55, 60, 65, 70, 75, 80, 85, 90, 95, 100, 105, 110, 115 or 120° C. In another embodiment, the endogenous enzymes inactivation is obtained by exposing the yeast cream at a temperature of no more than 120, 115, 110, 105, 100, 95, 90, 85, 80, 75, 70, 65, 60, 55, 50, 45, 40, 35, 30, 25, 20, 15, 10 or 5° C. In yet another embodiment, the endogenous enzymes inactivation is obtained by exposing the yeast cream at a temperature of between 5, 10, 15, 20, 25, 30, 35, 40, 45, 50, 55, 60, 65, 70, 75, 80, 85, 90, 95, 100, 105, 110, 115 or 120° C. and 120, 115, 110, 105, 100, 95, 90, 85, 80, 75, 70, 65, 60, 55, 50, 45, 40, 35, 30, 25, 20, 15, 10 or 5° C. In a further embodiment, the endogenous enzymes inactivation is obtained by exposing the yeast cream at a temperature between 5° and 110° C. In a yet further embodiment, the endogenous enzymes inactivation is obtained by exposing the yeast cream at a temperature between 6° and 100° C., for example, at a temperature of 80° C. In one embodiment, the endogenous enzymes inactivation is obtained by exposing the yeast cream at a certain pH or pH range. In an embodiment, the endogenous enzymes inactivation is obtained by exposing the yeast cream at a pH of between 1 and 13. In an embodiment, the endogenous enzymes inactivation is obtained by exposing the yeast cream at a pH of at least 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, 13.0 or higher. In another embodiment, the endogenous enzymes inactivation is obtained by exposing the yeast cream at a pH of no more than 13.0, 12.9, 12.8, 12.7, 12.6, 12.5, 12.4, 12.3, 12.2, 12.1, 12.0, 11.9, 11.8, 11.7, 11.6, 11.5, 11.4, 11.3, 11.2, 11.1, 11.0, 10.9, 10.8, 10.7, 10.6, 10.5, 10.4, 10.3, 10.2, 10.1, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1 or 1.0. In yet another embodiment, the endogenous enzymes inactivation is obtained by exposing the yeast cream at a pH between 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0 and 13.0, 12.9, 12.8, 12.7, 12.6, 12.5, 12.4, 12.3, 12.2, 12.1, 12.0, 11.9, 11.8, 11.7, 11.6, 11.5, 11.4, 11.3, 11.2, 11.1, 11.0, 10.9, 10.8, 10.7, 10.6, 10.5, 10.4, 10.3, 10.2, 10.1, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1 or 1.0. In a specific example, the endogenous enzymes inactivation is obtained by exposing the yeast cream at a pH below 5 or above 7, for example, at a pH of 3 or 9. In a specific example, the endogenous enzymes inactivation is obtained by exposing the yeast cream at a pH below 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1 or 1.0 or at a pH above 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0. In one embodiment, the endogenous enzymes inactivation is obtained by exposing the yeast cream to temperature and/or pH variations for a time higher than 1 second and lower than 10 hours. In an embodiment, the endogenous enzymes inactivation is obtained by exposing the yeast cream to temperature and/or pH variations for at least 15 s, at least 30 s, at least 45 s, at least 1 min, at least 2 min, at least 5 min, at least 10 min, at least 15 min, at least 20 min, at least 25 min, at least 30 min, at least 35 min, at least 40 min, at least 45 min, at least 50 min, at least 55 min, at least 1 h, at least 1.5 h, at least 2 h, at least 2.5 h, at least 3 h, at least 3.5 h, at least 4 h, at least 4.5 h, at least 5 h, at least 5.5 h, at least 6 h, at least 6.5 h, at least 7 h, at least 7.5 h, at least 8 h, at least 8.5 h, at least 9 h, at least 9.5 h, or at least 10 h. In another embodiment, the endogenous enzymes inactivation is obtained by exposing the yeast cream to temperature and/or pH variations for no more than 10 h, 9.5 h, 9 h, 8.5 h, 8 h, 7.5 h, 7 h, 6.5 h, 6 h, 5.5 h, 5 h, 4.5 h, 4 h, 3.5 h, 3 h, 2.5 h, 2 h, 1.5 h, 1 h, 55 min, 50 min, 45 min, 40 min, 35 min, 30 min, 25 min, 20 min, 15 min, 10 min, 5 min, 2 min, 1 min, 45 s, 30 s, or 15 s. In yet another embodiment, the endogenous enzymes inactivation is obtained by exposing the yeast cream at a temperature and/or pH variations for between 15 s, 30 s, 45 s, 1 min, 2 min, 5 min, 10 min, 15 min, 20 min, 25 min, 30 min, 35 min, 40 min, 45 min, 50 min, 55 min, 1 h, 1.5 h, 2 h, 2.5 h, 3 h, 3.5 h, 4 h, 4.5 h, 5 h, 5.5 h, 6 h, 6.5 h, 7 h, 7.5 h, 8 h, 8.5 h, 9 h, 9.5 h, or 10 h and 10 h, 9.5 h, 9 h, 8.5 h, 8 h, 7.5 h, 7 h, 6.5 h, 6 h, 5.5 h, 5 h, 4.5 h, 4 h, 3.5 h, 3 h, 2.5 h, 2 h, 1.5 h, 1 h, 55 min, 50 min, 45 min, 40 min, 35 min, 30 min, 25 min, 20 min, 15 min, 10 min, 5 min, 2 min, 1 min, 45 s, 30 s, or 15 s. In a further embodiment, the endogenous enzymes inactivation is obtained by exposing the yeast cream at a temperature and/or pH variations for between 15 s and 7 h. In a yet further embodiment, the endogenous enzymes inactivation is obtained by exposing the yeast cream at a temperature and/or pH variations for between 30 s and 2 h, for example, for 5 minutes. The one with ordinary skills in the art would know how to adapt the temperature, and/or the pH, and the exposure time according to the yeast source and to the endogenous enzymes to be inactivated. In a further embodiment, the endogenous enzymes inactivation can be confirmed by using standard techniques in the art such as, without being limited to, a protease activity assay (azoalbumin, Fluorescein, Thiocarbamoyl-Kappa-Casein or milk clot assay) and/or incubation of purified beta-glucans with the inactivated yeast cream and by measuring glucose formation (HPLC).

The inactivated yeast cream is then subjected to an exogenous enzymatic treatment. This is shown as step 030 in FIG. 1. In the context of the present disclosure, the exogenous enzymatic treatment lacks ribonuclease activity (i.e., endonuclease and exonuclease; (EC 3.1.4.1)). Still in the context of the present disclosure, the exogenous enzymatic treatment comprises at least one polypeptide capable of oligosaccharide hydrolysis. In one embodiment, the exogenous enzymatic treatment comprises at least one polypeptide having a glucanase, mannanase and/or chitinase activity. In an embodiment, the exogenous enzymatic treatment comprises at least one polypeptide having an endo-β-1,3-glucanase, exo-β-1,3-glucanase, endoβ-1,6-glucanase and/or exo-β-1,6-glucanase activity (EC 3.2.1). In another embodiment, the exogenous enzymatic treatment consists essentially of using a polypeptide having a β-1,3-glucanase activity and/or a polypeptide having a β-1,6-glucanase activity. In the context of the present disclosure, the expression “consists essentially of” excludes other enzymatic treatments that would materially affect protoplasts formation and/or the characteristics of the yeast proteins that can be obtained. In one embodiment, the exogenous enzymatic treatment comprising at least one polypeptide having a glucanase activity (EC 3.2.1) is performed at a temperature higher than 4° C. and lower than 120° C. In an embodiment, the exogenous enzymatic treatment comprising at least one polypeptide having a glucanase activity (EC 3.2.1) is performed at a temperature of at least 5, 10, 15, 20, 25, 30, 35, 40, 45, 50, 55, 60, 65, 70, 75, 80, 85, 90, 95, 100, 105, 110, 115 or 120° C. In another embodiment, the exogenous enzymatic treatment comprising at least one polypeptide having a glucanase activity (EC 3.2.1) is performed at a temperature of no more than 120, 115, 110, 105, 100, 95, 90, 85, 80, 75, 70, 65, 60, 55, 50, 45, 40, 35, 30, 25, 20, 15, 10 or 5° C. In yet another embodiment, the exogenous enzymatic treatment comprising at least one polypeptide having a glucanase activity (EC 3.2.1) is performed at a temperature of between 5, 10, 15, 20, 25, 30, 35, 40, 45, 50, 55, 60, 65, 70, 75, 80, 85, 90, 95, 100, 105, 110, 115 or 120° C. and 120, 115, 110, 105, 100, 95, 90, 85, 80, 75, 70, 65, 60, 55, 50, 45, 40, 35, 30, 25, 20, 15, 10 or 5° C. In a further embodiment, the exogenous enzymatic treatment comprising at least one polypeptide having a glucanase activity (EC 3.2.1) is performed at a temperature between 2° and 110° C. In a yet further embodiment, the exogenous enzymatic treatment comprising at least one polypeptide having a glucanase activity (EC 3.2.1) is performed at a temperature between 5° and 80° C., for example, at a temperature of 60° C. In one embodiment, the amount of polypeptide having a glucanase activity (EC 3.2.1) is higher than 1 UI and lower than 5000 UI per kg of inactivated yeast cream. In the context of the present disclosure, one unit of enzymatic activity (UI) catalyzes the formation of one μmol of glucose per min at 37° C. In an embodiment, the amount of polypeptide having a glucanase activity (EC 3.2.1) is at least 1, 5, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 UI per kg of inactivated yeast cream. In another embodiment, the amount of polypeptide having a glucanase activity (EC 3.2.1) is no more than 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100, 90, 80, 70, 60, 50, 40, 30, 20, 10, 5, or 1 UI per kg of inactivated yeast cream. In another embodiment, the amount of polypeptide having a glucanase activity (EC 3.2.1) is of between 1, 5, 10, 20, 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 and 5000, 4900, 4800, 4700, 4600, 4500, 4400, 4300, 4200, 4100, 4000, 3900, 3800, 3700, 3600, 3500, 3400, 3300, 3200, 3100, 3000, 2900, 2800, 2700, 2600, 2500, 2400, 2300, 2200, 2100, 2000, 1900, 1800, 1700, 1600, 1500, 1400, 1300, 1200, 1100, 1000, 900, 800, 700, 600, 500, 400, 300, 200, 100, 90, 80, 70, 60, 50, 40, 30, 20, 10, 5, or 1 UI per kg of inactivated yeast cream. In a further embodiment, the amount of polypeptide having a glucanase activity (EC 3.2.1) is of between 300 and 3000 UI per kg of inactivated yeast cream. In a yet further embodiment, the amount of polypeptide having a glucanase activity (EC 3.2.1) is of between 1500 and 2500 UI per kg of inactivated yeast cream, for example the amount of polypeptide having a glucanase activity (EC 3.2.1) is 2000 UI per kg of inactivated yeast cream. In one embodiment, the exogenous enzymatic treatment comprising at least one polypeptide having a glucanase activity (EC 3.2.1) is performed at a pH of between 3 and 9. In an embodiment, the exogenous enzymatic treatment comprising at least one polypeptide having a glucanase activity (EC 3.2.1) is performed at a pH of at least 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0 or higher. In another embodiment, the exogenous enzymatic treatment comprising at least one polypeptide having a glucanase activity (EC 3.2.1) is performed at a pH of no more than 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, or 3.0. In yet another embodiment, the exogenous enzymatic treatment comprising at least one polypeptide having a glucanase activity (EC 3.2.1) is performed at a pH of between 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0 and 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, or 3.0. In a further embodiment, the exogenous enzymatic treatment comprising at least one polypeptide having a glucanase activity (EC 3.2.1) is performed at a pH of between 4 and 8. In a yet further embodiment, the exogenous enzymatic treatment comprising at least one polypeptide having a glucanase activity (EC 3.2.1) is performed at a pH of between 5 and 7, for example, at a pH of 5.5. In one embodiment, the exogenous enzymatic treatment comprising at least one polypeptide having a glucanase activity (EC 3.2.1) is performed for a time higher than 1 second and lower than 10 hours. In an embodiment, the exogenous enzymatic treatment comprising at least one polypeptide having a glucanase activity (EC 3.2.1) is performed for at least 15 s, at least 30 s, at least 45 s, at least 1 min, at least 2 min, at least 5 min, at least 10 min, at least 15 min, at least 20 min, at least 25 min, at least 30 min, at least 35 min, at least 40 min, at least 45 min, at least 50 min, at least 55 min, at least 1 h, at least 1.5 h, at least 2 h, at least 2.5 h, at least 3 h, at least 3.5 h, at least 4 h, at least 4.5 h, at least 5 h, at least 5.5 h, at least 6 h, at least 6.5 h, at least 7 h, at least 7.5 h, at least 8 h, at least 8.5 h, at least 9 h, at least 9.5 h, at least 10 h, at least 11 h, at least 12 h, at least 13 h, at least 14 h, at least 15 h, at least 16 h, at least 17 h, at least 18 h, at least 19 h, at least 20 h, at least 21 h, at least 22 h, at least 23 h, or at least 24 h. In another embodiment, the exogenous enzymatic treatment comprising at least one polypeptide having a glucanase activity (EC 3.2.1) is performed for no more than 24 h, 23 h, 22 h, 21 h, 20 h, 19 h, 18 h, 17 h, 16 h, 15 h, 14 h, 13 h, 12 h, 11 h, 10 h, 9.5 h, 9 h, 8.5 h, 8 h, 7.5 h, 7 h, 6.5 h, 6 h, 5.5 h, 5 h, 4.5 h, 4 h, 3.5 h, 3 h, 2.5 h, 2 h, 1.5 h, 1 h, 55 min, 50 min, 45 min, 40 min, 35 min, 30 min, 25 min, 20 min, 15 min, 10 min, 5 min, 2 min, 1 min, 45 s, 30 s, or 15 s. In yet another embodiment, the exogenous enzymatic treatment comprising at least one polypeptide having a glucanase activity (EC 3.2.1) is performed for between 15 s, 30 s, 45 s, 1 min, 2 min, 5 min, 10 min, 15 min, 20 min, 25 min, 30 min, 35 min, 40 min, 45 min, 50 min, 55 min, 1 h, 1.5 h, 2 h, 2.5 h, 3 h, 3.5 h, 4 h, 4.5 h, 5 h, 5.5 h, 6 h, 6.5 h, 7 h, 7.5 h, 8 h, 8.5 h, 9 h, 9.5 h, 10 h, 11 h, 12 h, 13 h, 14 h, 15 h, 16 h, 17 h, 18 h, 19 h, 20 h, 21 h, 22 h, 23 h, or 24 h and 24 h, 23 h, 22 h, 21 h, 20 h, 19 h, 18 h, 17 h, 16 h, 15 h, 14 h, 13 h, 12 h, 11 h, 10 h, 9.5 h, 9 h, 8.5 h, 8 h, 7.5 h, 7 h, 6.5 h, 6 h, 5.5 h, 5 h, 4.5 h, 4 h, 3.5 h, 3 h, 2.5 h, 2 h, 1.5 h, 1 h, 55 min, 50 min, 45 min, 40 min, 35 min, 30 min, 25 min, 20 min, 15 min, 10 min, 5 min, 2 min, 1 min, 45 s, 30 s, or 15 s. In a further embodiment, the exogenous enzymatic treatment comprising at least one polypeptide having a glucanase activity (EC 3.2.1) is performed for between 1 and 24 h. In a yet further embodiment, the exogenous enzymatic treatment comprising at least one polypeptide having a glucanase activity (EC 3.2.1) is performed for between 2 h and 6 h, for example, for 4 h. The one with ordinary skills in the art would know how to adapt the polypeptide having a glucanase activity concentration, the temperature, the pH and the exposure time according to the yeast source and to the polypeptides having a glucanase activity considered.

The inactivated yeast cream is subjected to an enzymatic treatment to obtain yeast protoplasts. In the context of the present disclosure, the expression “yeast protoplast” refers to yeast that has been deprived from the majority of its cell wall components. In another embodiment, the enzymatic treatment of the methods of the present disclosure avoids using an enzyme having proteolytic activity and therefore aims at preserving the yeast protein's integrity (e.g., secondary, tertiary or quaternary structure). In some another embodiment, the enzymatic treatment of the present disclosure aims at preserving intracellular proteins as well as yeast cell membrane proteins integrity. In a further embodiment, the enzymatic treatment of the present disclosure aims at solubilizing the yeast cell wall, leaving a soluble fraction comprising mannoproteins and/or β-glucans and an insoluble fraction comprising yeast protoplasts. In yet another embodiment, the enzymatic treatment of the present disclosure allows solubilizing some of the yeast's membrane but does not allow the diffusion of the yeast proteins outside the yeast protoplast.

In some embodiments, prior to the enzyme treatment step 030, the inactivated yeast cream can be submitted to an alkaline extraction step 025. The alkaline extraction step can be conducted to reduce the amount of nucleotides (including polynucleotides such as RNA) in the final composition. In some embodiments, the alkaline extraction step can be conducted under conditions allowing the extraction of the nucleotides from the inactivated yeast cream and preventing or limiting the degradation of the nucleotides from the inactivated yeast cream. The expression “preventing or limiting the degradation of the nucleotides from the inactivated yeast cream” refers to the fact that the alkaline extraction step prevents or limit the degradation of the nucleotides by at least 50%. Without wishing to be bound to theory, it is understood that submitting the inactivated yeast cream to an alkaline extraction solubilizes, at least in part, the nucleotides present in the inactivated yeast cream while allowing the formation of yeast protoplasts (after the exogenous enzymatic treatment). The alkaline extraction step 025 comprises placing the inactivated yeast cream in alkaline conditions within a certain temperature range and for a range of time so as to allow the solubilization of nucleotides (including polynucleotides like RNA) outside the cells of the inactivated yeast cream. In an embodiment, the alkaline extraction step 025 is performed at a pH of between 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9 and 12.0, 11.9, 11.8, 11.7, 11.6, 11.5, 11.4, 11.3, 11.2, 11.1, 11.0, 10.9, 10.8, 10.7, 10.6, 10.5, 10.4, 10.3, 10.2, 10.1, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, or 8.1. In a further embodiment, the alkaline extraction step 025 is performed at a pH of between 8 and 11. In a yet further embodiment, the alkaline extraction step 025 is performed at a pH of between 8.5 and 9.5, for example, at a pH of 9.0. In additional embodiments, the alkaline extraction step 025 is performed at a temperature higher than 4° C. and lower than 70° C. In an embodiment, the alkaline extraction step 025 is performed at a temperature of at least 4, 5, 10, 15, 20, 25, 30, 35, 40, 45, 50, 55, 60, 65, or 70° C. In another embodiment, the alkaline extraction step is performed at a temperature of no more than 70, 65, 60, 55, 50, 45, 40, 35, 30, 25, 20, 15, 10, 5 or 4° C. In yet another embodiment, the alkaline extraction step 025 is performed at a temperature of between 4, 5, 10, 15, 20, 25, 30, 35, 40, 45, 50, 55, 60, 65, or 70° C. and 70, 65, 60, 55, 50, 45, 40, 35, 30, 25, 20, 15, 10, 5 or 4° C. In a further embodiment, the alkaline extraction step is performed at a temperature between 5° and 70° C., for example, at a temperature of 65° C. In yet another embodiment, the alkaline extraction step 025 is performed for between 15 s, 30 s, 45 s, 1 min, 2 min, 5 min, 10 min, 15 min, 20 min, 25 min, 30 min, 35 min, 40 min, 45 min, 50 min, 55 min, 1 h, 1.5 h, 2 h, 2.5 h, 3 h, 3.5 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h and 24 h, 23 h, 22 h, 21 h, 20 h, 19 h, 18 h, 17 h, 16 h, 15 h, 14 h, 13 h, 12 h, 11 h, 10 h, 9 h, 8 h, 7 h, 6 h, 5 h, 4 h, 3.5 h, 3 h, 2.5 h, 2 h, 1.5 h, 1 h, 55 min, 50 min, 45 min, 40 min, 35 min, 30 min, 25 min, 20 min, 15 min, 10 min, 5 min, 2 min, 1 min, 45 s, or 30 s. In a further embodiment, the alkaline extraction step 025 is performed for between 1 and 4 h. In a yet further embodiment, the alkaline extraction step 025 is performed for between 1.5 h and 2.5 h, for example, for 2 h. In yet another embodiment, the alkaline extraction step 025 is performed for between 15 s, 30 s, 45 s, 1 min, 2 min, 5 min, 10 min, 15 min, 20 min, 25 min, 30 min, 35 min, 40 min, and 45 min and 45 min, 40 min, 35 min, 30 min, 25 min, 20 min, 15 min, 10 min, 5 min, 2 min, 1 min, 45 s, or 30 s. In a further embodiment, the alkaline extraction step 025 is performed for between 10 and 45 min. In a yet further embodiment, the alkaline extraction step 025 is performed for between 20 min and 40 min, for example, for 30 min. The one with ordinary skills in the art would know how to optimize the temperature, the pH and the exposure time of the alkaline extraction step to solubilize the nucleotides from the inactivated yeast cream.

The method can include, in some embodiments, a step for inactivating the exogenous enzymes used during the enzyme treatment step 030 (not shown on FIG. 1).

The insoluble fraction obtained upon the enzymatic treatment performed in the method herein is then separated from the soluble fraction by usual techniques known by the one with ordinary skills in the art. This is shown as step 050 of FIG. 1. The techniques that can be used include, without being limited to, sedimentation of the insoluble fraction, centrifugation and/or filtration. In one embodiment, the insoluble fraction obtained is directly submitted to separation. In an embodiment, once separated from the soluble fraction, the insoluble fraction is optionally rinsed and then collected. In another embodiment, once separated from the soluble fraction, the insoluble fraction is directly collected. In one embodiment, the collected insoluble fraction has a neutral taste and can be in a non-dried or dried state. In the context of the present disclosure, the non-dried insoluble fraction can be in a semi-liquid state.

In one embodiment, the insoluble fraction can be provided in a non-dried state. In one embodiment, the non-dried insoluble fraction can be used straight away after having been collected to be formulated in an edible composition. In an embodiment, the non-dried insoluble fraction can be stored. In another embodiment, the non-dried insoluble fraction can be frozen according to the usual methods of the art in order to be formulated at a later date in an edible composition. In a further embodiment, the non-dried insoluble fractions obtained from different yeast sources can be combined in order to be formulated in an edible composition. In one embodiment, the insoluble fraction can be provided in a dried state. In another embodiment, the dried insoluble fraction is obtained by, without being limited to, spray drying, fluid bed drying, tray draying, roller/drum drying, infra red drying, or freeze drying/lyophilization of the non-dried insoluble fraction. In another embodiment, the dried insoluble fraction can be stored. In yet another embodiment, the dried insoluble fraction can be stored in order to be formulated at a later date in an edible composition. In a further embodiment, dried insoluble fractions obtained from different yeast sources can be combined in order to be formulated in an edible composition.

In one embodiment, the collected insoluble fraction comprising yeast protoplasts, when dried, has a protein content of higher than 50% and lower than 100% based on the total mass of the dried collected insoluble fraction, as determined by the Kjeldahl method for nitrogen analysis with 6.25 as conversion factor. In an embodiment, the collected insoluble fraction comprising yeast protoplasts, when dried, has a protein content of at least 50, 52, 54, 56, 58, 60, 62, 64, 66, 68, 70, 72, 74, 76, 78, 79, 80, 81, 82, 83, 84, 85, 86, 87, 88, 89, 90, 91, 92, 93, 94, 95, 96, 97, 98, or 99% based on the total mass of the dried collected insoluble fraction, as determined by the Kjeldahl method for nitrogen analysis with 6.25 as conversion factor. In another embodiment, the collected insoluble fraction comprising yeast protoplasts, when dried, has a protein content of no more than 100, 99, 98, 97, 96, 95, 94, 93, 92, 91, 90, 89, 88, 87, 86, 85, 84, 83, 82, 81, 80, 79, 78, 76, 74, 72, 70, 68, 66, 64, 62, 60, 58, 56, 54, 52, or 50% based on the total mass of the dried collected insoluble fraction, as determined by the Kjeldahl method for nitrogen analysis with 6.25 as conversion factor. In yet another embodiment, the collected insoluble fraction comprising yeast protoplasts, when dried, has a protein content of between 50, 52, 54, 56, 58, 60, 62, 64, 66, 68, 70, 72, 74, 76, 78, 79, 80, 81, 82, 83, 84, 85, 86, 87, 88, 89, 90, 91, 92, 93, 94, 95, 96, 97, 98, 99 or 100% and 100, 99, 98, 97, 96, 95, 94, 93, 92, 91, 90, 89, 88, 87, 86, 85, 84, 83, 82, 81, 80, 79, 78, 76, 74, 72, 70, 68, 66, 64, 62, 60, 58, 56, 54, 52, or 50% based on the total mass of the dried collected insoluble fraction, as determined by the Kjeldahl method for nitrogen analysis with 6.25 as conversion factor. In a further embodiment, the collected insoluble fraction comprising yeast protoplasts, when dried, has a protein content of between 70 and 100% based on the total mass of the dried collected insoluble fraction, as determined by the Kjeldahl method for nitrogen analysis with 6.25 as conversion factor. In a yet further embodiment, the collected insoluble fraction comprising yeast protoplasts, when dried, has a protein content of between 75 and 85%, for example, a protein content of 80% based on the total mass of the dried collected insoluble fraction, as determined by the Kjeldahl method for nitrogen analysis with 6.25 as conversion factor. In still another embodiment, the collected insoluble fraction comprising yeast protoplasts, when dried, has a protein content of at least 80% based on the total mass of the dried collected insoluble fraction, as determined by the Kjeldahl method for nitrogen analysis with 6.25 as conversion factor

The collected insoluble fraction comprising yeast protoplasts, when dried, can also have at least one of the following characteristics:
a lipid content of higher than 0% and lower than 20% based on the total mass of the dried collected insoluble fraction, as determined by the modified Mojonnier method (AOAC 989.05); Alternatively, a lipid content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11.0, 12.0, 13.0, 14.0, 15.0, 16.0, 17.0, 18.0, 19.0, or 20.0% based on the total mass of the dried collected insoluble fraction, as determined by the modified Mojonnier method (AOAC 989.05); Alternatively, a lipid content of no more than 20.0, 19.0, 18.0, 17.0, 16.0, 15.0, 14.0, 13.0, 12.0, 11.0, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction, as determined by the modified Mojonnier method (AOAC 989.05); Alternatively, a lipid content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11.0, 12.0, 13.0, 14.0, 15.0, 16.0, 17.0, 18.0, 19.0, or 20.0 and 20.0, 19.0, 18.0, 17.0, 16.0, 15.0, 14.0, 13.0, 12.0, 11.0, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction, as determined by the modified Mojonnier method (AOAC 989.05); Alternatively, a lipid content of between 6 and 10% based on the total mass of the dried collected insoluble fraction, as determined by the modified Mojonnier method (AOAC 989.05); Alternatively, a lipid content of between 7 and 9, for example a lipid content of 8% based on the total mass of the dried collected insoluble fraction, as determined by the modified Mojonnier method (AOAC 989.05);
when no alkaline extraction step has been performed, a nucleic acid content higher than 2% and lower than 30% based on the total mass of the dried collected insoluble fraction; Alternatively, a nucleic acid content of at least 2, 3, 4, 5, 6, 7, 8, 9, 10, 11, 12, 13, 14, 15, 16, 17, 18, 19, 20, 21, 22, 23, 24, 25, 26, 27, 28, 29 or 30% based on the total mass of the dried collected insoluble fraction; Alternatively, a nucleic acid content of no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2% based on the total mass of the dried collected insoluble fraction; Alternatively, a nucleic acid content of between 2, 3, 4, 5, 6, 7, 8, 9, 10, 11, 12, 13, 14, 15, 16, 17, 18, 19, 20, 21, 22, 23, 24, 25, 26, 27, 28, 29 or 30% and 30, 29, 28, 27, 26, 25, 24, 23, 22, 21, 20, 19, 18, 17, 16, 15, 14, 13, 12, 11, 10, 9, 8, 7, 6, 5, 4, 3, or 2% based on the total mass of the dried collected insoluble fraction; Alternatively, a nucleic acid content of between 5 and 20% based on the total mass of the dried collected insoluble fraction; Alternatively, a nucleic acid content of between 7 and 15%, for example a nucleic acid content of 10% based on the total mass of the dried collected insoluble fraction. In the context of the present disclosure, the expression “nucleic acid” refers to biopolymers (i.e., polynucleotides) composed of nucleotides, the latter being composed of a 5-carbon sugar, a phosphate group and a nitrogenous base. Still in the context of the present disclosure, the expression “nucleic acid” refers to polynucleotides such as deoxyribonucleic acid (DNA) and/or ribonucleic acid (RNA). Still in the context of the present disclosure, the expression “nucleic acid” refers to ribonucleic acid (RNA). In the context of the present disclosure, the term “nucleic acid” does not refer to nucleotide residues that may have been generated during the process. In the present disclosure, the nucleic acid content is measured according to the method of Fish et al. (1991), comparing values with and without enzymatic digestion;
when an alkaline extraction step has been performed, a nucleic acid content equal to or lower than 3% based on the total mass of the dried collected insoluble fraction. Alternatively, a nucleic acid content equal to or lower than 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.2 or 0.1% based on the total mass of the dried collected insoluble fraction. Alternatively, a nucleic acid content between 0.1 and 3% based on the total mass of the dried collected insoluble fraction. Alternatively, a nucleic acid content of between 0.5 and 2% based on the total mass of the dried collected insoluble fraction. In the context of the present disclosure, the expression “nucleic acid” refers to biopolymers (i.e., polynucleotides) composed of nucleotides, the latter being composed of a 5-carbon sugar, a phosphate group and a nitrogenous base. Still in the context of the present disclosure, the expression “nucleic acid” refers to polynucleotides such as deoxyribonucleic acid (DNA) and/or ribonucleic acid (RNA). Still in the context of the present disclosure, the expression “nucleic acid” refers to ribonucleic acid (RNA). In the context of the present disclosure, the term “nucleic acid” does not refer to nucleotide residues that may have been generated during the process. In the present disclosure, the nucleic acid content is measured according to the method of Fish et al. (1991), comparing values with and without enzymatic digestion;
a carbohydrate content higher than 0% and lower than 25%; Alternatively, a carbohydrate content of at least 0.5, 1, 2, 3, 4, 5, 6, 7, 8, 9, 10, 11, 12, 13, 14, 15, 16, 17, 18, 19, 20, 21, 22, 23, 24, or 25% based on the total mass of the dried collected insoluble fraction; Alternatively, a carbohydrate content of no more than 25, 24, 23, 22, 21, 20, 19, 18, 17, 16, 15, 14, 13, 12, 11, 10, 9, 8, 7, 6, 5, 4, 3, 2, 1, or 0.5% based on the total mass of the dried collected insoluble fraction; Alternatively, a carbohydrate content of between 0.5, 1, 2, 3, 4, 5, 6, 7, 8, 9, 10, 11, 12, 13, 14, 15, 16, 17, 18, 19, 20, 21, 22, 23, 24, or 25% and 25, 24, 23, 22, 21, 20, 19, 18, 17, 16, 15, 14, 13, 12, 11, 10, 9, 8, 7, 6, 5, 4, 3, 2, 1, or 0.5% based on the total mass of the dried collected insoluble fraction; Alternatively, a carbohydrate content of between 5 and 20% based on the total mass of the dried collected insoluble fraction; Alternatively, a carbohydrate content of between 7 and 15%, for example a carbohydrate content of 10%, based on the total mass of the dried insoluble fraction. In the context of the present disclosure, the term “carbohydrate” refers to total sugars measured by HPLC after chemical or enzymatic digestion (i.e., AOAC 980.13 method);
a mannan content of higher than 0% and lower than 6% based on the total mass of the dried collected insoluble fraction; Alternatively, a mannan content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0% based on the total mass of the dried collected insoluble fraction; Alternatively, a mannan content of no more than 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a mannan content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, or 5.9 and 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a mannan content of between 0 and 5.5% or of between 0 and 4% based on the total mass of the dried collected insoluble fraction; Alternatively, a mannan content of between 0.5 and 5.5 or of between 0.5 and 4.0, for example a mannan content of about 2% or 5% based on the total mass of the dried collected insoluble fraction. In the context of the present disclosure, the mannan content is measured by HPLC after chemical or enzymatic digestion;
a glucan content of higher than 0% and lower than 10% based on the total mass of the dried collected insoluble fraction; Alternatively, a glucan content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% based on the total mass of the dried collected insoluble fraction; Alternatively, a glucan content of no more than 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a glucan content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 and 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a glucan content of between 0 and 8% based on the total mass of the dried collected insoluble fraction; Alternatively, a glucan content of between 1 and 4, for example a glucan content of 2% based on the total mass of the dried collected insoluble fraction. In the context of the present disclosure, the glucan content is measured by HPLC after chemical or enzymatic digestion; and/or
a glucose content higher than 0% and lower than 25%; Alternatively, a glucose content of at least 0.5, 1, 2, 3, 4, 5, 6, 7, 8, 9, 10, 11, 12, 13, 14, 15, 16, 17, 18, 19, 20, 21, 22, 23, 24, or 25% based on the total mass of the dried collected insoluble fraction; Alternatively, a glucose content of no more than 25, 24, 23, 22, 21, 20, 19, 18, 17, 16, 15, 14, 13, 12, 11, 10, 9, 8, 7, 6, 5, 4, 3, 2, 1, or 0.5% based on the total mass of the dried collected insoluble fraction; Alternatively, a glucose content of between 0.5, 1, 2, 3, 4, 5, 6, 7, 8, 9, 10, 11, 12, 13, 14, 15, 16, 17, 18, 19, 20, 21, 22, 23, 24, or 25% and 25, 24, 23, 22, 21, 20, 19, 18, 17, 16, 15, 14, 13, 12, 11, 10, 9, 8, 7, 6, 5, 4, 3, 2, 1, or 0.5% based on the total mass of the dried collected insoluble fraction; Alternatively, a glucose content of between 5 and 20% based on the total mass of the dried collected insoluble fraction; Alternatively, a glucose content of between 7 and 15%, for example a glucose content of 10% based on the total mass of the dried insoluble fraction. In the context of the present disclosure, the term “glucose” refers to a monosaccharide having the molecular formula C₆H₁₂O₆. Still in the context of the present disclosure, glucose content is directly measured by HPLC.

In some embodiments, the method can include obtaining a soluble fraction of the enzymatically treated yeast cream. This corresponds to step 040 of FIG. 1.

### Yeast Protoplasts Derived Composition

In one embodiment, the present disclosure aims at providing a composition comprising yeast proteins derived from yeast protoplasts. In an embodiment, the composition comprising yeast proteins derived from yeast protoplasts has at least one of the following characteristics:
a protein content of higher than 50% and lower than 100% based on the total mass of the composition, as determined by the Kjeldahl method for nitrogen analysis with 6.25 as conversion factor. Alternatively, a protein content of at least 50, 52, 54, 56, 58, 60, 62, 64, 66, 68, 70, 72, 74, 76, 78, 79, 80, 81, 82, 83, 84, 85, 86, 87, 88, 89, 90, 91, 92, 93, 94, 95, 96, 97, 98, 99 or 100% based on the total mass of the composition, as determined by the Kjeldahl method for nitrogen analysis with 6.25 as conversion factor. Alternatively, a protein content of no more than 100, 99, 98, 97, 96, 95, 94, 93, 92, 91, 90, 89, 88, 87, 86, 85, 84, 83, 82, 81, 80, 79, 78, 76, 74, 72, 70, 68, 66, 64, 62, 60, 58, 56, 54, 52, or 50% based on the total mass of the composition, as determined by the Kjeldahl method for nitrogen analysis with 6.25 as conversion factor. Alternatively, a protein content of between 50, 52, 54, 56, 58, 60, 62, 64, 66, 68, 70, 72, 74, 76, 78, 79, 80, 81, 82, 83, 84, 85, 86, 87, 88, 89, 90, 91, 92, 93, 94, 95, 96, 97, 98, 99 or 100% and 100, 99, 98, 97, 96, 95, 94, 93, 92, 91, 90, 89, 88, 87, 86, 85, 84, 83, 82, 81, 80, 79, 78, 76, 74, 72, 70, 68, 66, 64, 62, 60, 58, 56, 54, 52, or 50% based on the total mass of the composition, as determined by the Kjeldahl method for nitrogen analysis with 6.25 as conversion factor. Alternatively, a protein content of between 70 and 100% based on the total mass of the composition, as determined by the Kjeldahl method for nitrogen analysis with 6.25 as conversion factor. Alternatively, a protein content of between 75 and 85%, for example, a protein content of 80% based on the total mass of the composition, as determined by the Kjeldahl method for nitrogen analysis with 6.25 as conversion factor;
a lipid content higher than 0% and lower than 20% based on the total mass of the dried collected insoluble fraction, as determined by the modified Mojonnier method (AOAC 989.05); Alternatively, a lipid content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11.0, 12.0, 13.0, 14.0, 15.0, 16.0, 17.0, 18.0, 19.0, or 20.0% based on the total mass of the dried collected insoluble fraction, as determined by the modified Mojonnier method (AOAC 989.05); Alternatively, a lipid content of no more than 20.0, 19.0, 18.0, 17.0, 16.0, 15.0, 14.0, 13.0, 12.0, 11.0, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction, as determined by the modified Mojonnier method (AOAC 989.05); Alternatively, a lipid content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11.0, 12.0, 13.0, 14.0, 15.0, 16.0, 17.0, 18.0, 19.0, or 20.0 and 20.0, 19.0, 18.0, 17.0, 16.0, 15.0, 14.0, 13.0, 12.0, 11.0, 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction, as determined by the modified Mojonnier method (AOAC 989.05); Alternatively, a lipid content of between 6 and 10% based on the total mass of the dried collected insoluble fraction, as determined by the modified Mojonnier method (AOAC 989.05); Alternatively, a lipid content of between 7 and 9, for example a lipid content of 8% based on the total mass of the dried collected insoluble fraction, as determined by the modified Mojonnier method (AOAC 989.05);
when no alkaline extraction step has been performed, a nucleic acid content higher than 2% and lower than 30% based on the total mass of the dried collected insoluble fraction; Alternatively, a nucleic acid content of at least 2, 3, 4, 5, 6, 7, 8, 9, 10, 11, 12, 13, 14, 15, 16, 17, 18, 19, 20, 21, 22, 23, 24, 25, 26, 27, 28, 29 or 30% based on the total mass of the dried collected insoluble fraction; Alternatively, a nucleic acid content of no more than 30, 29, 28, 27, 26, 25, 24, 23, 22, 21, 20, 19, 18, 17, 16, 15, 14, 13, 12, 11, 10, 9, 8, 7, 6, 5, 4, 3, or 2% based on the total mass of the dried collected insoluble fraction; Alternatively, a nucleic acid content of between 2, 3, 4, 5, 6, 7, 8, 9, 10, 11, 12, 13, 14, 15, 16, 17, 18, 19, 20, 21, 22, 23, 24, 25, 26, 27, 28, 29 or 30% and 30, 29, 28, 27, 26, 25, 24, 23, 22, 21, 20, 19, 18, 17, 16, 15, 14, 13, 12, 11, 10, 9, 8, 7, 6, 5, 4, 3, or 2% based on the total mass of the dried collected insoluble fraction; Alternatively, a nucleic acid content of between 5 and 20% based on the total mass of the dried collected insoluble fraction; Alternatively, a nucleic acid content of between 7 and 15%, for example a nucleic acid content of 10% based on the total mass of the dried collected insoluble fraction. In the context of the present disclosure, the expression “nucleic acid” refers to biopolymers (i.e., polynucleotides) composed of nucleotides, the latter being composed of a 5-carbon sugar, a phosphate group and a nitrogenous base. Still in the context of the present disclosure, the expression “nucleic acid” refers to polynucleotides such as deoxyribonucleic acid (DNA) and/or ribonucleic acid (RNA). Still in the context of the present disclosure, the expression “nucleic acid” refers to ribonucleic acid (RNA). In the context of the present disclosure, the term “nucleic acid” does not refer to nucleotide residues that may have been generated during the process. In the present disclosure, the nucleic acid content is measured according to the method of Fish et al. (1991), comparing values with and without enzymatic digestion;
when an alkaline extraction step has been performed, a nucleic acid content equal to or lower than 3% based on the total mass of the dried collected insoluble fraction. Alternatively, a nucleic acid content equal to or lower than 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.2 or 0.1% based on the total mass of the dried collected insoluble fraction. Alternatively, a nucleic acid content between 0.1 and 3% based on the total mass of the dried collected insoluble fraction. Alternatively, a nucleic acid content of between 0.5 and 2.0% based on the total mass of the dried collected insoluble fraction. In the context of the present disclosure, the expression “nucleic acid” refers to biopolymers (i.e., polynucleotides) composed of nucleotides, the latter being composed of a 5-carbon sugar, a phosphate group and a nitrogenous base. Still in the context of the present disclosure, the expression “nucleic acid” refers to polynucleotides such as deoxyribonucleic acid (DNA) and/or ribonucleic acid (RNA). Still in the context of the present disclosure, the expression “nucleic acid” refers to ribonucleic acid (RNA). In the context of the present disclosure, the term “nucleic acid” does not refer to nucleotide residues that may have been generated during the process. In the present disclosure, the nucleic acid content is measured according to the method of Fish et al. (1991), comparing values with and without enzymatic digestion;
a carbohydrate content higher than 0% and lower than 25%; Alternatively, a carbohydrate content of at least 0.5, 1, 2, 3, 4, 5, 6, 7, 8, 9, 10, 11, 12, 13, 14, 15, 16, 17, 18, 19, 20, 21, 22, 23, 24, or 25% based on the total mass of the composition; Alternatively, a carbohydrate content of no more than 25, 24, 23, 22, 21, 20, 19, 18, 17, 16, 15, 14, 13, 12, 11, 10, 9, 8, 7, 6, 5, 4, 3, 2, 1, or 0.5% based on the total mass of the composition; Alternatively, a carbohydrate content of between 0.5, 1, 2, 3, 4, 5, 6, 7, 8, 9, 10, 11, 12, 13, 14, 15, 16, 17, 18, 19, 20, 21, 22, 23, 24, or 25% and 25, 24, 23, 22, 21, 20, 19, 18, 17, 16, 15, 14, 13, 12, 11, 10, 9, 8, 7, 6, 5, 4, 3, 2, 1, or 0.5% based on the total mass of the composition; Alternatively, a carbohydrate content of between 5 and 20% based on the total mass of the composition; Alternatively, a carbohydrate content of between 7 and 15%, for example a carbohydrate content of 10%, based on the total mass of the composition. In the context of the present disclosure, the term “carbohydrate” refers to total sugars measured by HPLC after chemical or enzymatic digestion (i.e., AOAC 980.13 method);
a mannan content of higher than 0% and lower than 6% based on the total mass of the dried collected insoluble fraction; Alternatively, a mannan content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9 or 6.0% based on the total mass of the dried collected insoluble fraction; Alternatively, a mannan content of no more than 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a mannan content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.1, 5.2, 5.3, 5.4, 5.5, 5.6, 5.7, 5.8, 5.9 or 6.0 and 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a mannan content of between 0 and 5.5% or between 0 and 4.0% based on the total mass of the dried collected insoluble fraction; Alternatively, a mannan content of between 0.5 and 5.5% or of between 0.5 and 3.0, for example a mannan content of about 2% or about 5.5% based on the total mass of the dried collected insoluble fraction. In the context of the present disclosure, the mannan content is measured by HPLC after chemical or enzymatic digestion;
a glucan content of higher than 0% and lower than 10% based on the total mass of the dried collected insoluble fraction; Alternatively, a glucan content of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% based on the total mass of the dried collected insoluble fraction; Alternatively, a glucan content of no more than 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a glucan content of between 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0 and 10.0, 9.9, 9.8, 9.7, 9.6, 9.5, 9.4, 9.3, 9.2, 9.1, 9.0, 8.9, 8.8, 8.7, 8.6, 8.5, 8.4, 8.3, 8.2, 8.1, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5, 0.4, 0.3, 0.2, or 0.1% based on the total mass of the dried collected insoluble fraction; Alternatively, a glucan content of between 0 and 8% based on the total mass of the dried collected insoluble fraction; Alternatively, a glucan content of between 1 and 4, for example a glucan content of 2% based on the total mass of the dried collected insoluble fraction. In the context of the present disclosure, the glucan content is measured by HPLC after chemical or enzymatic digestion; and/or
a glucose content higher than 0% and lower than 25%; Alternatively, a glucose content of at least 0.5, 1, 2, 3, 4, 5, 6, 7, 8, 9, 10, 11, 12, 13, 14, 15, 16, 17, 18, 19, 20, 21, 22, 23, 24, or 25% based on the total mass of the composition; Alternatively, a glucose content of no more than 25, 24, 23, 22, 21, 20, 19, 18, 17, 16, 15, 14, 13, 12, 11, 10, 9, 8, 7, 6, 5, 4, 3, 2, 1, or 0.5% based on the total mass of the composition; Alternatively, a glucose content of between 0.5, 1, 2, 3, 4, 5, 6, 7, 8, 9, 10, 11, 12, 13, 14, 15, 16, 17, 18, 19, 20, 21, 22, 23, 24, or 25% and 25, 24, 23, 22, 21, 20, 19, 18, 17, 16, 15, 14, 13, 12, 11, 10, 9, 8, 7, 6, 5, 4, 3, 2, 1, or 0.5% based on the total mass of the composition; Alternatively, a glucose content of between 5 and 20% based on the total mass of the composition; Alternatively, a glucose content of between 7 and 15%, for example a glucose content of 10% based on the total mass of the composition. In the context of the present disclosure, the term “glucose” refers to a monosaccharide having the molecular formula C₆H₁₂O₆. Still in the context of the present disclosure, glucose content is directly measured by HPLC.

In an embodiment, the yeast proteins derived from yeast protoplasts are intracellular yeast proteins, yeast cell membrane proteins, or a combination thereof.

In an embodiment, the composition comprising yeast proteins derived from yeast protoplasts having a neutral taste. This is in contrast with yeast proteins obtained from a yeast extract (especially those obtained with using a protease treatment) which usually exhibit a medium taste. In the context of the present disclosure, the yeast proteins derived from yeast protoplasts of an edible product have a neutral taste and can thus be used as ingredients whose taste does not affect/modify the taste of the other components of said composition. Hence, the composition of the present disclosure does not affect/modify the taste of a formulated edible product. In another embodiment, the composition comprising yeast proteins derived from yeast protoplasts having a neutral taste when compared to plant/leguminous protein compositions. In another embodiment, the composition comprising yeast proteins derived from yeast protoplasts having a neutral taste when compared to yeast protein composition derived from a method lacking a glucanase treatment step. In yet another embodiment, the composition comprising yeast proteins derived from yeast protoplasts having a neutral taste when compared to compositions lacking yeast protoplasts. In the context of the present disclosure, a method for preparing a yeast protein composition, the method lacking a glucanase treatment step, is a method which comprises of i) providing a yeast cream comprising yeasts; ii) inactivating the endogenous enzymes of the yeasts to provide an inactivated yeast cream; iii) optionally separating the insoluble fraction from the soluble fraction and iv) collecting the inactivated yeast cream or optionally collecting the inactivated yeast cream insoluble fraction, excluding a glucanase treatment step. Still in the context of the present disclosure, a method for preparing a yeast protein composition, the method lacking a glucanase treatment step is a method which consists essentially of i) providing a yeast cream comprising yeasts; ii) inactivating the endogenous enzymes of the yeasts to provide an inactivated yeast cream; iii) optionally separating the insoluble fraction from the soluble fraction and iv) collecting the inactivated yeast cream or optionally collecting the inactivated yeast cream insoluble fraction. The transitional phrase “consisting essentially of” limits the method steps to the specified steps “(i.e., no glucanase treatment step and no insoluble/soluble fraction separation step) and those that do not materially affect the basic and novel characteristic(s)” of the final product.

In one embodiment, the composition comprising yeast proteins derived from yeast protoplasts has a water solubility index higher than 1% and lower than 15%, based on the total mass of the dried composition. In an embodiment, the composition comprising yeast proteins derived from yeast protoplasts has a water solubility index higher than 1, 2, 3, 4, 5, 6, 7, 8, 9, 10, 11, 12, 13, 14, or 15% based on the total mass of the dried composition. In another embodiment, the composition comprising yeast proteins derived from yeast protoplasts has a water solubility index of no more than 15, 14, 13, 12, 11, 10, 9, 8, 7, 6, 5, 4, 3, 2, or 1% based on the total mass of the dried composition. In yet another embodiment, the composition comprising yeast proteins derived from yeast protoplasts has a water solubility index of between 1, 2, 3, 4, 5, 6, 7, 8, 9, 10, 11, 12, 13, 14, or 15% and 15, 14, 13, 12, 11, 10, 9, 8, 7, 6, 5, 4, 3, 2, or 1%, based on the total mass of the dried composition. In a further embodiment, the composition comprising yeast proteins derived from yeast protoplasts has a water solubility index of between 1 and 10%, based on the total mass of the dried composition. In a yet further embodiment, the composition comprising yeast proteins derived from yeast protoplasts has a water solubility index of between 3 and 7%, based on the total mass of the dried composition, for example a water solubility index of 5. In the context of the present disclosure, the water solubility index presents the weight of dry solids in the supernatant expressed as a percentage of the original weight of sample. In another embodiment, the composition comprising yeast proteins derived from yeast protoplasts has a water solubility index lower compared to yeast protein composition prepared with a similar method lacking a glucanase treatment step.

In one embodiment, the composition comprising yeast proteins derived from yeast protoplasts has weak emulsifying properties. In the context of the present disclosure, the expression emulsifying properties refers to emulsification activity and/or emulsion stability of said composition. Still in the context of the present disclosure, the expression “emulsification activity” refers to the ability of the composition to form an emulsion, whereas the expression “emulsion stability” refers to the remaining emulsion after a heat treatment. In an embodiment, the composition comprising yeast proteins derived from yeast protoplasts has between 1 and 10 times less emulsification activity than lecithin, when measured with the Brishti et al. (2017) method. In another embodiment, the composition comprising yeast proteins derived from yeast protoplasts has at least 1, 2, 3, 4, 5, 6, 7, 8, 9, or 10 times less emulsification activity than lecithin, when measured with the Brishti et al. (2017) method. In another embodiment, the composition comprising yeast proteins derived from yeast protoplasts has no more than 10, 9, 8, 7, 6, 5, 4, 3, 2, or 1 times less emulsification activity than lecithin, when measured with the Brishti et al. (2017) method. In yet another embodiment, the composition comprising yeast proteins derived from yeast protoplasts has between 1, 2, 3, 4, 5, 6, 7, 8, 9, or 10 and 10, 9, 8, 7, 6, 5, 4, 3, 2, or 1 times less emulsification activity than lecithin, when measured with the Brishti et al. (2017) method. In a further embodiment, the composition comprising yeast proteins derived from yeast protoplasts has between 1 and 10 times less emulsification activity than lecithin, when measured with the Brishti et al. (2017) method. In a yet further embodiment, the composition comprising yeast proteins derived from yeast protoplasts has between 3 and 7 times less emulsification activity than lecithin, for example has 5 times less emulsification activity than lecithin, when measured with the Brishti et al. (2017) method. In an embodiment, the composition comprising yeast proteins derived from yeast protoplasts has between 1 and 15 times less emulsion stability than lecithin, when measured with the Yasumatsu et al. (1972) method. In another embodiment, the composition comprising yeast proteins derived from yeast protoplasts has at least 1, 2, 3, 4, 5, 6, 7, 8, 9, 10, 11, 12, 13, 14 or 15 times less emulsion stability than lecithin, when measured with the Yasumatsu et al. (1972) method. In another embodiment, the composition comprising yeast proteins derived from yeast protoplasts has no more than 15, 14, 13, 12, 11, 10, 9, 8, 7, 6, 5, 4, 3, 2, or 1 times less emulsion stability than lecithin, when measured with the Yasumatsu et al. (1972) method. In yet another embodiment, the composition comprising yeast proteins derived from yeast protoplasts has between 1, 2, 3, 4, 5, 6, 7, 8, 9, 10, 11, 12, 13, 14, or 15 and 15, 14, 13, 12, 11, 10, 9, 8, 7, 6, 5, 4.3, 2, or 1 times less emulsion stability than lecithin, when measured with the Yasumatsu et al. (1972) method. In a further embodiment, the composition comprising yeast proteins derived from yeast protoplasts has between 5 and 15 times less emulsion stability than lecithin, when measured with the Yasumatsu et al. (1972) method. In a yet further embodiment, the composition comprising yeast proteins derived from yeast protoplasts has between 8 and 12 times less emulsion stability than lecithin, for example the composition has 10 times less emulsion stability than lecithin, when measured with the Yasumatsu et al. (1972) method.

In one embodiment, the composition comprising yeast proteins derived from yeast protoplasts has weak foaming properties. In the context of the present disclosure, the expression “foaming properties” refers to foam capacity and/or foam stability over time. Still in the context of the present disclosure, the expression “foam capacity” of the composition refers to the amount of interfacial area that can be created by said composition, whereas the expression “foam stability” refers to the ability of the composition to stabilize against gravitational and mechanical stresses over time. In an embodiment, the composition comprising yeast proteins derived from yeast protoplasts has between 1 and 10 times less foam capacity than yeast protein composition prepared with a similar method lacking a glucanase treatment step, when measured with the method described in Chandra et al. (2015). In another embodiment, the composition comprising yeast proteins derived from yeast protoplasts has at least 1, 2, 3, 4, 5, 6, 7, 8, 9, or 10 times less foam capacity than yeast protein composition prepared with a similar method lacking a glucanase treatment step, when measured with the method described in Chandra et al. (2015). In another embodiment, the composition comprising yeast proteins derived from yeast protoplasts has no more than 10, 9, 8, 7, 6, 5, 4, 3, 2, or 1 times less foam capacity than yeast protein composition prepared with a similar method lacking a glucanase treatment step, when measured with the method described in Chandra et al. (2015). In a yet another embodiment, the composition comprising yeast proteins derived from yeast protoplasts has between 1, 2, 3, 4, 5, 6, 7, 8, 9, or 10 and 10, 9, 8, 7, 6, 5, 4, 3, 2, or 1 times less foam capacity than yeast protein composition prepared with a similar method lacking a glucanase treatment step, when measured with the method described in Chandra et al. (2015). In a further embodiment, the composition comprising yeast proteins derived from yeast protoplasts has between 1 and 6 times less foam capacity than yeast protein composition prepared with a similar method lacking a glucanase treatment step, when measured with the method described in Chandra et al. (2015). In a yet further embodiment, the composition comprising yeast proteins derived from yeast protoplasts has between 1 and 4 times less foam capacity than yeast protein composition prepared with a similar method lacking a glucanase treatment step, for example the composition has 3 times less foam capacity than yeast protein composition prepared with a similar method lacking a glucanase treatment step, when measured with the method described in Chandra et al. (2015). In an embodiment, the composition comprising yeast proteins derived from yeast protoplasts has between 1 and 10 times less foam stability than yeast protein composition prepared with a method lacking a glucanase treatment step, when measured after 30 minutes with the method described in Brishti et al. (2017). In another embodiment, the composition comprising yeast proteins derived from yeast protoplasts has at least 1, 2, 3, 4, 5, 6, 7, 8, 9, or 10 times less foam stability than yeast protein composition prepared with a method lacking a glucanase treatment step, when measured after 30 minutes with the method described in Brishti et al. (2017). In another embodiment, the composition comprising yeast proteins derived from yeast protoplasts has no more than 10, 9, 8, 7, 6, 5, 4, 3, 2, or 1 times less foam stability than yeast protein composition prepared with a method lacking a glucanase treatment step, when measured after 30 minutes with the method described in Brishti et al. (2017). In yet another embodiment, the composition comprising yeast proteins derived from yeast protoplasts has between 1, 2, 3, 4, 5, 6, 7, 8, 9, or 10 and 10, 9, 8, 7, 6, 5, 4, 3, 2, or 1 times less foam stability than yeast protein composition prepared with a method lacking a glucanase treatment step, when measured after 30 minutes with the method described in Brishti et al. (2017). In a further embodiment, the composition comprising yeast proteins derived from yeast protoplasts has between 3 and 9 times less foam stability than yeast protein composition prepared with a method lacking a glucanase treatment step, when measured after 30 minutes with the method described in Brishti et al. (2017). In a yet further embodiment, the composition comprising yeast proteins derived from yeast protoplasts has between 4 and 8 times less foam stability than yeast protein composition prepared with a method lacking a glucanase treatment step.

In one embodiment, the composition comprising yeast proteins derived from yeast protoplasts has a lower viscosity than 12.2 mPa·s at room temperature, as determined by the Onwulata et al. (2014) method. In one another embodiment, the composition comprising yeast proteins derived from yeast protoplasts has a viscosity of about 10 mPa·s at room temperature, as determined by the Onwulata et al. (2014) method. In an embodiment, the composition comprising yeast proteins derived from yeast protoplasts has a lower viscosity than 8.5 mPa·s when heated at 85° C. for 5 min, as determined by the Onwulata et al. (2014) method. In another embodiment, the composition comprising yeast proteins derived from yeast protoplasts has a viscosity of about 7.3 mPa·s when heated at 85° C. for 5 min, as determined by the Onwulata et al. (2014) method. In a further embodiment, the composition comprising yeast proteins derived from yeast protoplasts has a lower viscosity than 14.1 mPa·s when cooled down to room temperature after having been heated at 85° C. for 5 min, as determined by the Onwulata et al. (2014) method. In a yet further embodiment, the composition comprising yeast proteins derived from yeast protoplasts has a viscosity of about 11.2 mPa·s when cooled down to room temperature after having been heated at 85° C. for 5 min, as determined by the Onwulata et al. (2014) method.

In one embodiment, the composition comprising yeast proteins derived from yeast protoplasts has a Protein Digestibility-Corrected Amino Acid Score (PDCAAS) higher than yeast protein composition prepared with a method lacking a glucanase treatment step. For example, in some embodiments, the PDCAAS of the composition of the present disclosure can be equal to or higher than 1.0. In an embodiment, the composition comprising yeast proteins derived from yeast protoplasts is more digest than yeast protein composition prepared with a method lacking a glucanase treatment step.

In one embodiment, the composition comprising yeast proteins derived from yeast protoplasts is obtainable or obtained by the method for obtaining a composition having yeast proteins disclosed herein. In one another embodiment, the composition comprising yeast proteins derived from yeast protoplasts comprises the collected insoluble fraction comprising yeast protoplasts of the method for obtaining a composition having yeast proteins disclosed herein. In yet another embodiment, the composition comprising yeast proteins derived from yeast protoplasts consists essentially of the collected insoluble fraction comprising yeast protoplasts of the method for obtaining a composition having yeast proteins disclosed herein. In a further embodiment, the compositions comprising yeast proteins derived from yeast protoplasts obtained from different yeast sources can be combined in order to be formulated in an edible composition.

### Edible Product

In one embodiment, the present disclosure aims at providing an edible product. In an embodiment, the edible product comprises yeast proteins and at least one further ingredient. In another embodiment, the edible product comprises a composition comprising yeast proteins derived from yeast protoplasts and at least one further ingredient. In yet another embodiment, the composition comprised in the edible product is the composition comprising yeast proteins derived from yeast protoplasts of the present disclosure, and at least one further ingredient.

In an embodiment, the composition comprised in the edible product provides between 1 and 100%, based on the total mass of the edible product, of the proteins of the edible product. In another embodiment, the composition comprised in the edible product provides at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%, based on the total mass of the edible product, of the proteins of the edible product. In yet another embodiment, the composition comprised in the edible product provides no more than 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%, based on the total mass of the edible product, of the proteins of the edible product. In some embodiment, the composition comprised in the edible product provides between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% and 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%, based on the total mass of the edible product, of the proteins of the edible product. In the context of the present disclosure, the proportion of the composition within the edible product would depend on the nature of the edible product. In some specific embodiment, the proportion of the composition in a cheese analogue can be of between 2-4% based on the total mass of the cheese analogue product. In some another specific embodiment, the proportion of the composition in a protein bar can be of between 30-40% based on the total mass of the protein bar. In a further embodiment, the composition of the present disclosure can be consumed as is (i.e., flakes), then the proportion of the composition would be close to 100%. The one with ordinary skills in the art would know how to adapt the composition proportions depending on the edible product considered. In an embodiment, the edible product can be liquid or solid. In another embodiment, the edible product can be a beverage ingredient, a beverage product, a food ingredient, a food product, a feed ingredient, and/or a feed product. In some embodiment, the edible product can be a beverage ingredient and/or a beverage product. In some further embodiment, the edible product can be, without being limited to, a shake, a dairy substitute such as milk substitute, a liquid meal, a soup, a broth, a smoothie, a cream, a gravy, etc. In some embodiment, the edible product can be a food ingredient and/or a food product. In some further embodiment, the edible product can be a baked product, a cooked/boiled product and/or a dairy-like product. In a yet further embodiment, the baked product can be, without being limited to, a bread, a cake, a muffin, a cookie, a pita, a tortilla, a bun, a flatbread, a brownie, a cracker, a pastry, a pie, a tarte, a tort, a pizza, etc. In a yet further embodiment, the cooked/boiled product can be, without being limited to, a bar, flakes, breakfast cereals, a meat product such as bacon, sausage, hamburger/chopped/minced steak, a meat alternative product, a plant-based meat, pastas, noodles, a seasoning, a snack, a confectionary, gummies, a chocolate-based product, a ready meal product, etc. In a yet further embodiment, the dairy-like product can be, without being limited to, yogurt, cheese, cheese alternative product, cream, butter, custard, ice cream, etc. In some embodiment, the edible product can be a feed ingredient and/or a feed product. In some another embodiment, the feed ingredient/product can be a concentrate, a roughage, or a mixed feed. In a yet further embodiment, the feed ingredient/product can be, without being limited to, baked kibbles, biscuits, meals, extruded products, loaf, chunks, chunk-in-loaf products, pellets, crumbles, etc.

The present disclosure provides a method for making an edible product. The method comprises combining the composition of the present disclosure with at least one ingredient to obtain an edible product. The method can include heating the composition and the at least one ingredient to obtain the edible product (e.g., a heated edible product). The method can include baking the composition and the at least one ingredient to obtain the edible product (e.g., a baked edible product). The method can include blending the composition and the at least one ingredient to obtain the edible product (e.g., a blended edible product). The method can include freezing the composition and the at least one ingredient to obtain the edible product (e.g., a frozen edible product). The method can include extruding the composition and the at least one ingredient to obtain the edible product (e.g., an extruded edible product). The method can include fermenting the composition and the at least one ingredient to obtain the edible product (e.g., a fermented edible product).

### Use of the Edible Product

In one embodiment, the present disclosure aims at using of the edible product of the present disclosure for human and/or animal nutrition. In an embodiment, the edible product provides as an alternative to animal or plant/leguminous proteins-based edible products. In another embodiment, the edible product is combined to animal or plant/leguminous proteins-based edible products. In yet another embodiment, the edible product is consumed/administered orally.

In an embodiment, the edible product of the present disclosure is intended to be combined with at least one food ingredient, at least one feed ingredient, and/or at least one beverage ingredient. The yeast composition can be, in some embodiments, included directly in a food product, a feed product and/or a beverage product. In such embodiments, the yeast composition can be admixed with a feed additive, a food additive, a further beverage additive and/or a binder. When the edible product of the present disclosure is intended to be used as an additive, it can be provided in a liquid form, which can be, in some further embodiments, a spray-dryable liquid form. Alternatively, or in combination, the edible product can be provided in a powder form, which can, in some further embodiments, be a free-flowing powder.

In an embodiment, the edible product of the present disclosure is used as a food/feed supplement. In another embodiment, the food/feed supplement is enterally administered and is intended to supplement a diet by increasing the total dietary intake, or a concentrate, a metabolite, a constituent, and/or an extract of a subject compared to a non-supplemented subject receiving the same diet. As such, the food/feed supplement can be co-administered per os with, without being limited to, vitamins, minerals, essential fatty acids, natural products, and/or probiotics. When the edible product of the present disclosure is intended to be used as a supplement, it can be provided in a liquid form, which can be, in some further embodiments, an oil, a solution or a spray-dryable liquid form. Alternatively, or in combination, the supplement can be provided in a powder form which can be, in some further embodiments, a capsule, a pill, a tablet, a confectionary, a gummy, etc.

In an embodiment, the edible product disclosed herein is for wellness, weight control, for elderly and/or for sports applications of human and/or animals. In another embodiment, the edible product herein can be incorporated into a diet or a restrictive diet. In the context of the present disclosure, the term “diet” is understood to refer to any kind and amount of food and drink that is ingested by the subject (i.e., human and/or animal). Still in the context of the present disclosure, the expression “restrictive diet” refers to restriction imposed on a subject's diet (i.e., human and/or animal's diet) to limit the kind and/or the amount of food that is prescribed to be ingested. In another embodiment, the edible product when administered for a restrictive diet can be used to reduce the subject's overall weight and/or fat mass, to increase the subject's lean mass, muscle mass, average power, endurance, and/or to modulate the subject's hormonal balance.

In an embodiment, the edible product disclosed herein can be used as a meal replacement and/or a medical food product. In another embodiment, the edible product of the present disclosure can be used to limit, to avoid or replace the use of animal protein in edible products' formulations. In another embodiment, the edible product is intended to be used for illness, injury and/or surgery recovery. In another embodiment, the edible product disclosed herein, is intended to be used for oral/enteral clinical nutrition.

### EXAMPLES

### Example 1: Processes for Making Various Compositions Comprising Yeast Proteins from Saccharomyces

The process for making the various compositions is schematically provided in FIG. 1. Composition A (obtained after step 020 in FIG. 1) corresponds to a composition having yeast proteins prepared according to the following method. Briefly: Saccharomyces cerevisiae yeasts were cultured following a standard fed-batch fermentation protocol, washed and separated via centrifugation to obtain a yeast cream. The yeast endogenous enzymes were inactivated with a heat exchanger in a holding tube by heating the yeast cream at 95° C. for 4 min. The resultant slurry was then spray dried, giving the Composition A. Composition B (obtained after step 050 in FIG. 1) corresponds to a composition having yeast proteins. Briefly: Saccharomyces cerevisiae yeasts were cultured to obtain a yeast cream as described above. The yeast endogenous enzymes were inactivated by heating the yeast cream at 95° C. for 4 min. The resultant slurry was then cool down to 60° C. and the pH was adjusted to pH 5.5 prior to be subjected to a β-1,3-glucanase treatment (i.e., Denazyme GEL™, Nagase; 0.5% on a yeast dry matter basis) for at least 4 h at 60° C. and 5.5. The enzymatic reaction was stopped by heating the hydrolysate to 95° C. for at least 2 min (this step also served as a pasteurization step). The resultant insoluble and soluble fractions were separated by centrifugation, and the insoluble fraction was collected and then spray dried at 85° C., giving the Composition B. The soluble fraction obtained therefrom (obtained after step 040 in FIG. 1) was also spray dried giving the Composition C.

### Example 2: Comparison of Compositions A, B and C

Protein content (via Kjeldahl method of nitrogen analysis with 6.25 as coefficient), lipid content (via modified Mojonnier method; AOAC 989.05), nucleic acid content (via Fish et al. (1991) method; Comparing values with and without enzymes digestion of the nucleic acid), total carbohydrate content (via monosaccharides titration with HPLC after enzymatic/chemical digestion; AOAC 980.13), mannan content (via mannose titration with HPLC after enzymatic/chemical digestion), glucan content (via glucose titration with HPLC after enzymatic/chemical digestion), and glucose content (via direct glucose titration with HPLC) were determined for Compositions A, B and C. These compositions (i.e., A, B and C) were obtained from Example 1 and were characterized in the example herein according to standard methods known by the one with ordinary skills in the art. The characterization results are provided in Table 1. Briefly, Composition B contains more proteins but less total carbohydrate than Compositions A and C. This observation is in line with the Composition B microscopy analyses revealing protoplasts formation. (data not shown). Neither lipid nor nucleic acid content has been reported for Composition C.

TABLE 1
Characterisation results of Compositions A, B and
C. For each column, component percentage were
based on the total mass of the dried composition.

| | Composition A | Composition B | Composition C |
|---|---|---|---|
| | (no glucanase | (Separated | (Separated |
| | treatment, no | insoluble | soluble |
| Content | separation) | fraction) | fraction) |
| Protein (%) | 61.1 | 81.2 | 29.5 |
| Lipids (%) | 4.3 | 5.7 | — |
| Nucleic acids (%) | 8.0 | 10.5 | — |
| Total carbohydrate | 25 | 8.1 | 44.8 |
| (%) | | | |
| Glucans (%) | 15.3 | 5.4 | 32.2 |
| Mannans (%) | 9.7 | 2.3 | 13.7 |
| Glucose (%) | 15.3 | 5.4 | 32.2 |

### Example 3: Comparison of Compositions A and B

The protein contents of the compositions A and B obtained from the methods described in Example 1 were determined and compared using the Kjeldahl method of nitrogen analysis (i.e., standard method for organic substance protein content quantification). Briefly, both compositions A and B (0.25 g) were heated to 373° C. with concentrated sulfuric acid in the presence of copper sulfate as catalyst. Such oxidation reaction aims at decomposing the samples while releasing the reduced nitrogen as ammonium sulfate. The resultant solutions were then distilled with sodium hydroxide, releasing ammonia, which was further allowed to react with boric acid prior to be dissolved in distilled water. The alkaline products were titrated with hydrochloric acid. Both the Tashiro indicator and pH were used to determine the equivalence point (i.e., pH 4.9). Based on the titration results, a coefficient of 6.25 was used to determine the amount of nitrogen in the samples, allowing the protein content calculations.

The results of the Kjeldahl method of nitrogen analysis of both samples A and B are presented in Table 2 below. Composition A has a protein content of about 60% whereas Composition B has a protein content of about 80%, both based on the total mass of the analyzed composition (dried or non-dried). The sample subjected to a glucanase treatment (Composition B) has about 20% more protein content than its non-enzymatically treated homologue (Composition A).

TABLE 2
Kjeldahl method of nitrogen analysis of both composition
A and B results. Dwb % stands for % of protein content
based on the total dried composition mass.

| | | Calculated | | Protein | |
|---|---|---|---|---|---|
| Sample | Dry Weight | Protein | | content | |
| Name | % | Content % | Std Dev | Dwb % | Std Dev |
| A | 98.4 | 57.7 | 0.8 | 58.6 | 0.8 |
| B | 96.5 | 76.7 | 0.3 | 79.5 | 0.3 |

The water solubility index (WSI) of the two compositions having yeast proteins (i.e., A and B) were determined and compared herein. For each sample, 1 g of dried powder was suspended in 10 mL of distilled water. The resultant suspension was gently mixed for 30 min at room temperature prior to be centrifuged at 3000×g for 15 minutes at 25° C. The remaining supernatant was collected and allowed to decant, giving rise to a soluble fraction and some insoluble components. The soluble fraction was discarded, and the insoluble components were dried prior to be weighted. The WSI represents the mass of dry insoluble components expressed as a percentage of the original sample mass.

The results of the WSI determination of both samples A and B are presented in Table 3 below. Composition A has a WSI of about 23% whereas Composition B presents a WSI of about 5%, both based on the dry weight of the tested composition. The sample subjected to a glucanase treatment (Composition B) is about 5 times less soluble than its non-enzymatically treated homologue (Composition A).

TABLE 3
Water solubility index (WSI) of both samples A
and B results. Dwb % stands for % of protein content
based on the total dried composition mass.

| Sample Name | Dry Weight % | WSI Dwb % |
|---|---|---|
| A | 98.4 | 23.2 |
| B | 96.5 | 5.3 |

Both the emulsification activity and emulsion stability of the two compositions having yeast proteins (i.e., Compositions A and B) were determined and compared to lecithin (i.e., a standard emulsifier in the art). Emulsion activity (EA) is defined as the maximum amount of oil that can be emulsified per amount of dried composition. Emulsion stability (ES) is defined as the rate of phase separation in water and oil during emulsion storage. Both the EA and ES were determined using Brishti et al. (2017) and Yasumatsu et al. (1972) methods, respectively. Briefly: Each sample (i.e., Composition A, Composition B and lecithin; 0.24 g) was resuspended in 12 mL distilled water and 12 mL sunflower oil in a 50 mL centrifuge tube. The resultant mixtures were homogenized for 1 min with a homogenizer prior to be centrifuged at 1100×g for 5 minutes at 20° C.

The emulsification activity (EA) of both samples was calculated according to the following formula: EA=(H₁/H₀)×100; wherein H₁ refers to the measured emulsion volume and H₀ refers to the measured total volume of solution within the tube.

The emulsion stability (ES) of both samples was determined after i) heating the emulsion above at 80° C. for 30 min, ii) cooling the emulsion with tap water and iii) centrifugating the emulsion at 1100×g for 5 min at 20° C. ES was calculated using the following formula: ES=(H₁/H₀)×100; wherein H₁ refers to the measured emulsion volume and H₀ refers to the measured total volume of solution within the tube.

The results of the emulsification properties (i.e., EA and ES) are presented in Table 4 below. The EA of Composition A is comparable to that of lecithin whereas the sample subjected to a glucanase treatment (Composition B) presents 4 to 5 times less activity than both its non-enzymatically treated homologue (Composition A) and lecithin. The ES of Composition A is 3 to 4 times lower than lecithin but is 3 to 4 times higher than its enzymatically treated counterpart (Sample B). Hence, Composition B presents 10 to 11 times less emulsion stability than lecithin.

TABLE 4
Emulsification activity (EA) and emulsion stability (ES) results
of both compositions A and B, compared to lecithin (n = 2).

| | EA | EA Std Dev | ES | ES Std Dev |
|---|---|---|---|---|
| Sample Name | % | % | % | % |
| Sample A | 45.7 | 2.7 | 16.1 | 0.0 |
| Sample B | 11.5 | 0.0 | 4.5 | 1.9 |
| Lecithin | 50.4 | 1.2 | 51.4 | 0.8 |

Both the foaming capacity (FC) and foam stability (FS) of the two compositions having yeast proteins (i.e., Compositions A and B) were determined following the methods provided by Chandra et al. (2015) and Brishti et al. (2017), respectively. Briefly: each sample (Compositions A and Composition B; 0.2 g) was resuspended in 20 ml distilled water, within a 50 mL centrifugation tube. The suspension obtained therefrom was homogenized using a homogenizer and then whipped for 1 min prior to measurements. The foaming capacity (FC) of each sample was calculated according to the following formula: FC=[(V₂−V₁)/V₁]×100; wherein V₁ refers to the measured volume of the suspension prior to the whipping step and V₂ refers to the measured volume of the whipped suspension.

The foaming stability (FS) of each sample was measured over time (0, 15, 30, 45 and 60 min post suspension whipping) and was calculated according to the following formula: FS=(VFₜ/VFₜ₀)×100; wherein VFₜ refers to the measured foam volume at a specific time post whipping and VFₜ₀ refers to the measured foam volume right after whipping (i.e., 0 min).

The results of the foaming properties (i.e., FC and FS) are presented in Table 5 below. The sample subjected to a glucanase treatment (Composition B) has about 3 to 4 times less foaming capacity (FC) than its non-enzymatically treated homologue (Composition A). In other words, Composition A is 3 to 4 times more efficient at forming foam compared to its glucanase treated counterpart. The foaming stability studies revealed that the Composition A-based foam keeps about the half of its stability after 30 min whereas only 10% of the foam formed with the glucanase treated composition (Composition B) remains after the same time. Therefore, the foam derived from Composition B is about 4 to 5 times less stable than the foam prepared when the composition has not been subjected to a glucanase treatment (Composition A).

TABLE 5
Foaming capacity (FC) and foam stability (FS)
results of both samples A and B (N = 2).

| | | FC Std | | | FS(t) Std |
|---|---|---|---|---|---|
| Sample | FC | Dev | Time (t) | FS(t) | Dev |
| Name | % | % | min | % | % |
| Composition | 36.6 | 0.0 | 0 | 100.0 | 0.0 |
| A | | | 15 | 61.0 | 0.0 |
| | | | 30 | 50.6 | 4.3 |
| | | | 45 | 41.5 | 8.6 |
| | | | 60 | 32.9 | 0.0 |
| Composition | 9.6 | 3.7 | 0 | 100.0 | 0.0 |
| B | | | 15 | 16.4 | 3.0 |
| | | | 30 | 11.1 | 0.5 |
| | | | 45 | 0.0 | 0.0 |
| | | | 60 | 0.0 | 0.0 |

Both the smell and taste of the two compositions having yeast proteins (i.e., Composition A and B) were evaluated and compared to Nutralys® F85M (a pea protein isolate; Roquette) by trained assessors. The samples (Composition A, Composition B and Nutralys® F85M) were suspended in hot water to reach a 2% w/w suspension. The 2% w/w suspensions (40 mL) were served hot in a glass container to each assessor for organoleptic analysis (N=2, n=2).

The Rate-All-That-Apply (RATA) method was used to determine each sample organoleptic properties, allowing participants to select relevant terms from a given list and rate their intensity. During the assessment of the samples, two main organoleptic modalities were investigated, i.e., smell and taste. To do so, the following parameters were evaluated: overall intensity (mandatory), sour, cheesy, creamy, vegetable, chicken/poultry, beef, roasty, bready, fermented/alcoholic, and off-odor/taste. In addition, taste modality also included umami, salty, sweet, bitter, and astringent attributes. To measure the intensities, a numerical scale of 1 to 9 was applied, where 1 refers to “very low”; 5 to “moderate” and 9 to “very strong”. During the assessment, there was also a possibility to add comments on all modalities if desired.

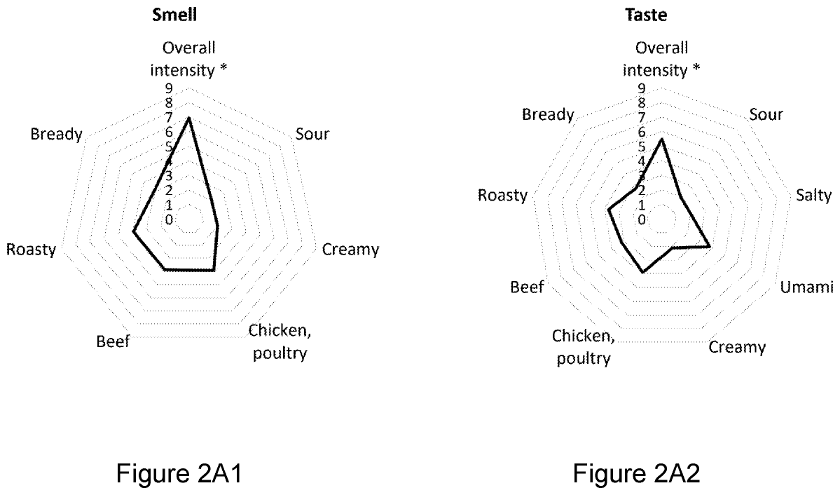

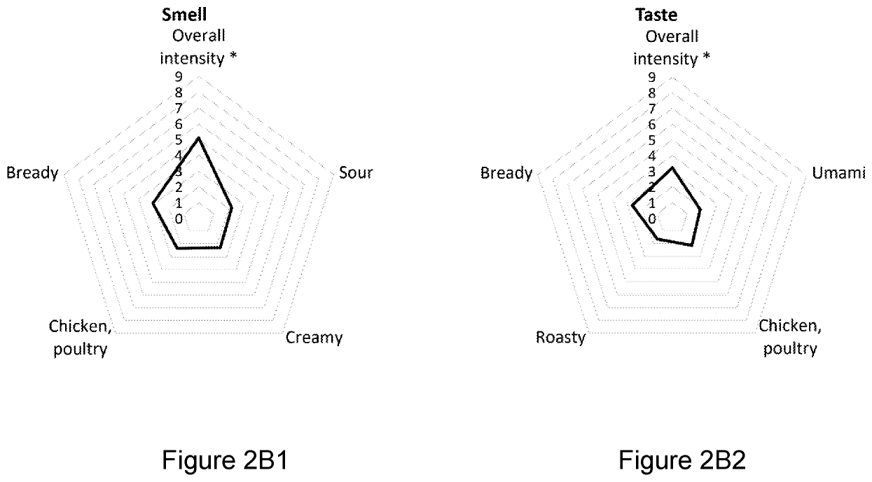

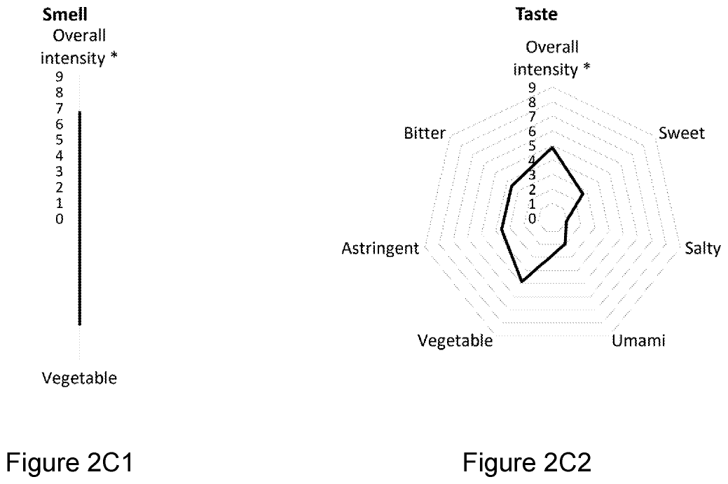

The results of these organoleptic assays are depicted in FIG. 2. Nutralys® F85M (FIG. 2C) is distinguishable by strong vegetable notes (pea odor and taste), bitterness and astringency. Composition A (FIG. 2A) was evaluated to have an intense smell and taste profiles with strong roast and beefiness along with chicken/poultry notes. Composition B (FIG. 2B) was overall much less intense and more neutral (i.e., presents the lowest average scores) than the other samples. None of the samples had cheesy (odor), fermented (taste), off-odor or off-taste, thus, these attributes were excluded from FIGS. 2A, 2B and 2C.

Viscosity measurements of both samples (Compositions A and B) were also performed according to Onwulata et al. (2014) method with slight modifications. Briefly: Each sample (2.7 g) was suspended in 27.3 mL ddH₂O, homogenized, and submitted for viscosity measurements using a rheometer Anton Paar Physica MCR301, program RheoCompass. RVA is performed using temperature ramp as a test type. The temperature profile implemented was as follows: 2 min at 25° C.→5 min 25-85° C. ramp linear→5 min at 85° C.→5 min 85-25° C. ramp linear→2 min at 25° C. The viscosity measurement results of both samples are presented in Table 6. Composition B is slightly less viscous than its non-enzymatically treated counterpart, regardless the condition tested.

TABLE 6
Viscosity measurements results of both compositions
A and B using 2 min at 25° C. → 5 min
25-85° C. ramp linear → 5 min at 85°
C. → 5 min 85-25° C. ramp linear →
2 min at 25° C. as temperature profile.

| | Initial | | End viscosity |
|---|---|---|---|
| | Viscosity at | Viscosity after | after cooling |
| | Room | heating at 85° C. | to Room |
| | temperature | for 5 min | temperature |
| Sample | (mPa · s) | (mPa · s) | (mPa · s) |
| Composition A | 12.2 | 8.5 | 14.1 |
| Composition B | 10.5 | 7.3 | 11.2 |

Protein digestibility-corrected amino acid score (PDCAAS) is a method of evaluating the quality of a protein based on both the amino acid requirements of humans and their ability to digest it. Hence, the PDCAAS method was performed to evaluate the digestibility of both compositions “A” and “B” (data not shown). The PDCAAS of the Composition B (which is higher than 1.0) is higher than the one of its non-enzymatically treated counterparts (i.e., Composition A).

### Example 4: Alkaline Extraction Optimization

It was determined if an alkaline extraction step could be used to extract nucleic acids and their derivatives from a yeast cream. Various pH and temperatures were used to determine their effect in solubilizing the nucleic acids outside the yeast cells.

A Saccharomyces cerevisiae yeast cream having an initial dry matter of 17.72% and an initial protein content of 60.95% was obtained. Samples (40 g) of yeast cream were pH adjusted to either 8.0, 9.0, 10.0 or 11.0. Each sample was incubated for 2 hours at different temperatures (4° C., room temperature, 55° C. or 65° C.) to generate the different samples.

Each was sample was centrifuged at 4500 rpm for 15 minutes and the supernatant was further characterized. No washing step was applied. The supernatants were spray dried. The DW of the supernatant was determined using a halogen dryer (Moisture analyzer, MA 37-1US, Sartorius). The alpha amino nitrogen (AAN), and ribonucleic acid (RNA) were determined using high pressure liquid chromatography.

Table 7 shows the AAN, and RNA yield found in the supernatant after the various alkaline extractions. As shown in Table 7, one of the compounds that is better extracted in alkaline conditions is RNA, as shown by the strong correlation between extraction pH and RNA recovery. The RNA content at 55° C. and 65° C. was considerably higher when compared to extractions at lower temperatures, reaching 23.5% in the extractions at 55° C. and pH 10 as well as at 65° C. and pH 9 (table 7). In the supernatants produced at 55° C./pH 11, 65° C./pH 10 and 65° C./pH 11, the RNA content was lower than in the lower pH extractions at the same temperature (table 7).

TABLE 7
AAN, and RNA yields in the supernatants of samples submitted
to alkaline extraction. Super. = supernatant,

| | | Super. | Super. | Super. | Super. | RNA |
|---|---|---|---|---|---|---|
| Temp. | | AAN | AAN | RNA | RNA | recovery |
| (° C.) | pH | (%) | (g) | (%) | (g) | (%) |
| 4° C. | 8 | 1.1 | 0.0031 | 0.8 | 0.0023 | 0.34% |
| | 9 | 1.0 | 0.0036 | 0.7 | 0.0025 | 0.37% |
| | 10 | 1.0 | 0.0035 | 0.9 | 0.0032 | 0.45% |
| | 11 | 1.1 | 0.0047 | 1.2 | 0.0053 | 0.79% |
| RT | 8 | 1.1 | 0.0035 | 0.8 | 0.0025 | 0.37% |
| | 9 | 1.1 | 0.0039 | 0.9 | 0.0032 | 0.46% |
| | 10 | 1.0 | 0.0042 | 1.0 | 0.0042 | 0.59% |
| | 11 | 1.1 | 0.0056 | 2.1 | 0.0107 | 1.54% |
| 55° C. | 8 | 2.6 | 0.0353 | 8.2 | 0.1115 | 18.56% |
| | 9 | 2.4 | 0.0323 | 16.5 | 0.2221 | 37.50% |
| | 10 | 2.0 | 0.0281 | 23.5 | 0.3306 | 56.94% |
| | 11 | 1.8 | 0.0318 | 17.8 | 0.3146 | 58.15% |
| 65° C. | 8 | 2.3 | 0.0337 | 18.7 | 0.2744 | 48.71% |
| | 9 | 2.1 | 0.0334 | 23.5 | 0.3738 | 64.94% |
| | 10 | 1.8 | 0.0308 | 19.9 | 0.3403 | 58.65% |
| | 11 | 1.5 | 0.0302 | 13.1 | 0.2642 | 46.59% |

### Example 5 Combination of Alkaline Extraction and Glucanase Treatment

It was then determined if an alkaline extraction step could be used, prior to a glucanase treatment, to reduce the nucleic acid content of the protein composition.

A Saccharomyces cerevisiae yeast cream having 16.0% solids, 63.47% protein, 4.06% phosphates and 7.90% RNA was collected. The yeast cream was heat inactivated at 95° C. for 5 min in an autolyser (Bailun, 20 L, 200 rpm), diluted to achieve 15% dry matter (DM) and submitted to an alkaline extraction process (65° C., pH 9.0, 2 hr). The sample obtained after alkaline treatment was analyzed for the presence of protoplasts (microscopy) and dry matter (DM) using a halogen dryer (Moisture analyzer, MA 37-1US, Sartorius). The sample obtained after alkaline extraction was then centrifuged at 4500 rpm, 10 minutes using a Sigma centrifuge (Model 4-5L, rotor: 11650) to generate a soluble fraction and an insoluble fraction. The insoluble fraction was washed 1× to a volume 1:1 and centrifuged again. The washed insoluble fraction was submitted to a glucanase treatment (Denazyme GEL, 60° C., pH 5.6, 5 h). The soluble fraction and the enzyme-treated insoluble fraction were spray dried and further analyzed.

The alpha amino nitrogen (AAN) was determined by a spectrophotometric method (adapted from EBC-Ninhydrin method for determination of free alpha amino nitrogen). The ribonucleic acid (RNA), glucans and mannans were determined using high pressure liquid chromatography. The protein yield was conducted using the Kjeldahl method. AN/TN ratio was calculated by dividing the AAN content by total nitrogen (e.g., protein content divided by 6.25).

After alkaline extraction and glucanase treatment, it was confirmed that protoplasts were present (data not shown). Under the experimental conditions tested, the use of an alkaline extraction step reduced the nucleic acid content below 2% (see Table 8) while maintaining the protein content above 80% (Table 9) in the yeast extract.

TABLE 8
RNA mass balance (% w/w) in various materials.

| | DM yield (%, | RNA yield (%, | RNA |
|---|---|---|---|
| | when compared | when compared | measured |
| Material | to yeast cream) | to yeast cream) | (%) |
| Yeast cream | 100.00 | 100.00 | 7.90 |
| Insoluble fraction | 72.89 | 0.88 | 1.20 |
| Soluble fraction | 27.11 | 74.81 | 21.80 |

TABLE 9
Characteristics of insoluble and soluble fractions.

| Characteristics | Insoluble fraction | Soluble fraction |
|---|---|---|
| Extraction yield (% when | 50.6 | 20.8 |
| compared to yeast cream) | | |
| Protein (% w/w) | 82.48 | 10.66 |
| Phosphates (% w/w) | 1.7 | 1.8 |
| RNA (% w/w) | 1.5 | 4.3 |
| Glucans (% w/w) | 0.6 | 35.6 |
| Mannans (% w/w) | 5.3 | 32.7 |

### Example 6: Composition B in a Vegan Cheese American Mozzarella Style Product

It was decided to characterize sensory and functional properties of a composition having yeast proteins prepared according to the method claimed in the present disclosure (i.e., Composition B, as in the examples above) in a vegan cheese type of products as a complement to pea protein. Two trials were performed: i) a pea protein based vegan cheese (3.59%) and ii) a partial pea protein substitution with “2.74% of the Composition B+0.85 pea protein” based vegan cheese, while all other components remained constant. The complete vegan cheese formulation is provided in Table 10 below. No coloring agent or opacifier have been included in the formulations.

TABLE 10
Firm shreddable mozzarella style block formulations.

| | | “Pea Protein + |
|---|---|---|
| | Pea Protein | Composition B” |
| | Vegan Cheese | Vegan Cheese |
| | Usage (%) | Usage (%) |
| Composition B | 0 | 2.74 |
| Vitessence Pulse 1853 Pea Protein | 3.59 | 0.85 |
| Ticagel ® CA 0957 | 16.52 | 16.52 |
| Sea salt | 1.73 | 1.73 |
| Water | 26.82 | 26.82 |
| Neutresca ® 51-25 | 17.49 | 17.49 |
| Sunflower oil | 6.28 | 6.28 |
| Oat milk | 26.82 | 26.82 |
| Natural Mozzarella Flavor 10-859- | 0.53 | 0.53 |
| 524-3 | | |
| Lactic Acid 60 FG PWD | 0.21 | 0.21 |
| Total (%) | 100 | 100 |

Color evaluation was performed by a visual observation analysis of the blocks and shreds. Pea protein vegan cheese presented a dairy yellowish hue, whereas its partially substituted counterpart (mix of Composition B and pea protein) was light earthy beige.

Sensory and texture properties were evaluated by cutting samples of both cheese analogues into 20 mm cubes after removal from the fridge (4° C.). Flavor and texture profiles were determined by trained assessors. The results are provided in Table 11 below.

TABLE 11
Flavor and texture characterization of the vegan Mozzarella
cheese analogues, i.e., Pea protein Vegan cheese and
“Pea protein + Composition B” vegan cheese.

| Cheese | | |
|---|---|---|
| Analogue | Flavor | Texture |
| Pea Protein | Clean, coconut notes, | Block with medium |
| Vegan Cheese | light beany notes, | firmness |
| | soft cheesy profile | Shredding possibilities |
| | | Slight granular texture |
| “Pea Protein + | Clean, neutral, salty, | The block was slightly |
| Composition B” | soft cheesy profile, | softer than its pea protein |
| Vegan Cheese | light beany notes | analogue, although |
| | | smoother, block shredded |
| | | similarly. |
| | | Slightly stickier on the |
| | | blades. |
| | | Shreds were bulkier with |
| | | less tendency to dry up at |
| | | cooking. |

The partial substitution of pea protein with a mixture of pea protein+Composition B did not impact the flavor profile (i.e., a neutral impact). However, the blocks prepared with the claimed composition were slightly softer than their pea protein analogues, although blocks shredded similarly.

### Example 6: High-Moisture Extrusion Cooking of Meat Analogs Containing the Composition B

It was decided to evaluate the effect of the addition of Composition B powder on plant-based meat analogs prepared using high-moisture extrusion cooking. During the high-moisture extrusion, the powder blend and water (about 50-70% of the total mass) were continuously dosed into the extruder, where the material was quickly heated up to 150° C., and the corotating screws mechanically mix and shear the mass. As a result, the dough transformed into a flowing melt. At the end of the barrel, the melt was pushed by the screws into a long cooling tunnel, which compacted the material and prevents water from boiling by cooling the mass below 100° C. and providing backpressure. The friction between the solidifying layers of the flowing material inside the cooling tunnel created long protein fibers, which resemble meat structures.

Different proportions of Composition B were added in dry blends as represented in Table 12, therefore partially replacing leguminous proteins (i.e., H5, H10, H15, H25 and H35). H0 does not contain any Composition B, whereas H100 contains Composition B only.

TABLE 12
Amount of the different proteins included in
the various samples of the meat analogues.

| | | | | Loryma | Lyckeby |
|---|---|---|---|---|---|
| | Composition | Caremoli | Aloja pea | wheat | potato |
| Samples | B | pea | 50.OBP | gluten | starch |
| H0 | — | 65 | 22 | 8 | 5 |
| H5 | 5 | 65 | 17 | 8 | 5 |
| H10 | 10 | 65 | 12 | 8 | 5 |
| H15 | 15 | 65 | 7 | 8 | 5 |
| H25 | 25 | 62 | — | 8 | 5 |
| H35 | 35 | 52 | — | 8 | 5 |
| H100 | 100 | — | — | — | — |

Meat analog extrudates were produced from all the tested blends as follows. The samples were processed in a co-rotating intermeshing twin-screw extruder KETSE 20/40 (Brabender GmbH, Duisburg, Germany) with a long cooling die (24×7×700 mm, W×H×L). The screw with a length-to-diameter ratio of 40 was configured to apply medium shear. The material mass flow rate was set constant to 4 kg h⁻¹ by calibrating the volumetric feeder with the powder. Water was added through a separate port by a calibrated peristaltic pump. The temperature profile was set to 45, 83-84, 135-137, and 151-154° C. Cooling die temperature was maintained at 65° C. with a tempering unit, but only in the last 300 mm, thus creating a temperature gradient along the die. Moisture content was adjusted for each blend and one sample was collected from each blend when the process reached stability as evidenced by the measured pressure and temperature. Screw speed and temperature were slightly adjusted between the blends to keep the process stable. The samples were packed into zip-lock bags and kept frozen at −20° C. until analysis.

Instrumental Texture Profile Analysis: Frozen samples were thawed and rehydrated for 2 h in 60° C. water (temperature was not maintained). Before the measurement, the pieces were blotted dry with a paper tissue. Texture profile analysis (TPA) was performed with TA.XTplusC Texture Analyzer (Stable Micro-Systems, Godalming, UK) equipped with a 75 mm flat probe and 5 kg load cell. The extruded pieces were cut into 15×15×7 mm shapes and compressed twice by 70% at 3 mm s⁻¹ probe speed, 1.5 mm s⁻¹ pre-test speed, and 1 s holding time between the compressions. The load cell was 50 kg. Hardness, chewiness, cohesiveness, springiness, and resilience were calculated by the texture analyzer software.

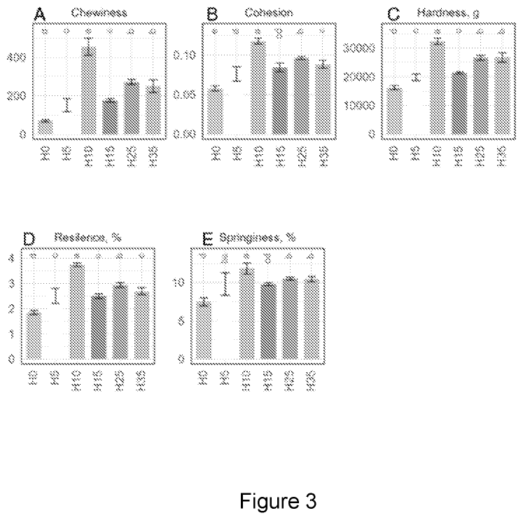

TPA of extrudates revealed the differences in the mechanical attributes of extrudates, which are summarized in FIG. 3. Sample H100 was not measured because its hardness was too high for the test equipment. All the tested extrudates parameters (i.e., hardness, chewiness, cohesiveness, springiness, and resilience) of the Composition B-based extrudates (H5, H10, H15, H25 and H35) were higher than the control (H0).

Descriptive Sensory Analysis (DSA): Frozen samples were thawed and rehydrated for 2 h in 60° C. water (temperature was not maintained). Before the sensory assessment, the pieces were blotted dry with a paper tissue and cut into 4 cm long pieces. The sensory analysis was conducted with eight expert assessors who had previous training and experience with such extruded plant-based samples. The analysis was performed in a standard (ISO 6668:2008) sensory room. The samples were coded with three-digit codes and served in cups. The order of the samples was randomized following the Williams Latin Square experimental design. Samples were served at room temperature. Water and crackers were served between samples for palate cleaning.

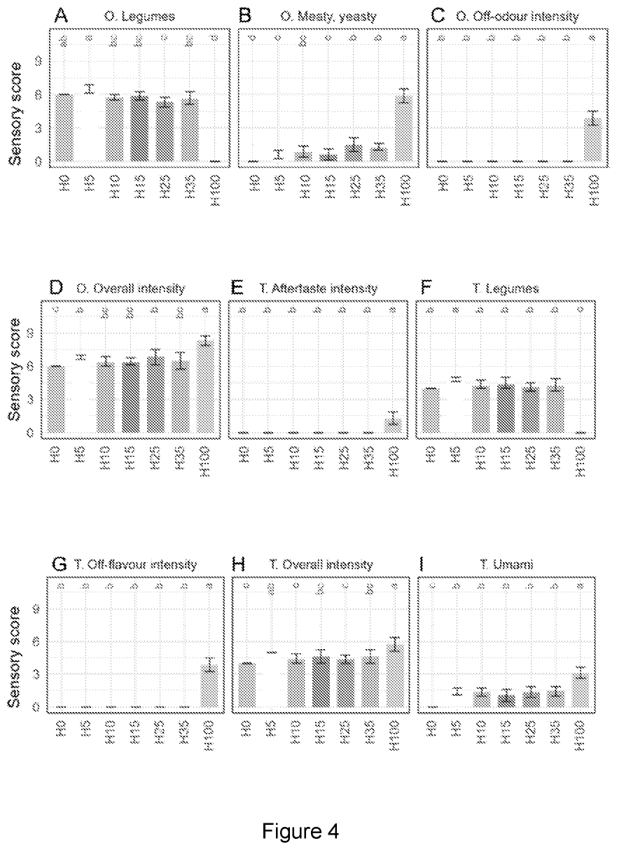

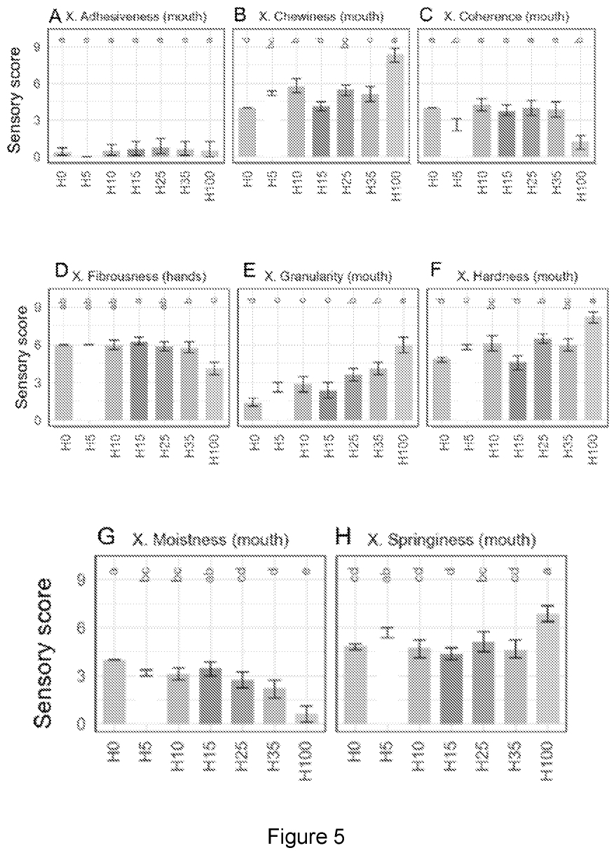

Samples were evaluated on a scale of 0-9 (“0”—none; “1”—very weak; “5”—moderate; “9”—very strong). Three modalities were evaluated in total: odor, taste, and texture. For odor and taste, the sensory analysis included attributes such as overall intensity, legumes, yeasty (odor)/umami (taste), and off-flavors. Aftertaste was additionally included for taste modality. The texture profile included the evaluation of fibrousness, springiness, hardness, chewiness, adhesiveness, granularity, coherence, and moistness. Only fibrousness was evaluated by hand, others by mouthfeel. Assessors had also the possibility to add comments to a voluntary text box for each modality. The DSA results for the different extrudates are depicted in FIG. 4 and FIG. 5. The observed differences were very small up to 35% yeast addition. But the 100% Composition B extrudate (H100) was noticeably different from the others according to all attributes. As shown in FIG. 4, Composition B has a mild flavor and its addition to samples H5-H35 increased meaty/yeasty odors and umaminess only slightly vs, Control (H0), but in H100 this increase was much larger. On the other hand, it was commented that H35 had roasty odor nuances, which may be related to the higher yeast content compared to H5-H25, although the meaty intensity was similar to H25. Legume odor and taste, and overall odor and taste intensity were not different in H0-H35, however, H100 had a stronger overall odor and taste. Off-odors, off-tastes, and aftertastes were detected only in H100. The off notes described for H100 included “dusty” and “papery”. As shown in FIG. 5, Texture attributes such as adhesiveness, coherence, fibrousness, and springiness showed no differences between samples H0-H35, except for H100, which was more coherent and springier, but less fibrous. Other attributes had more apparent differences: chewiness, hardness, and granularity were elevated in samples H5-H35 vs. Control (H0).

### Example 7: Processes for Making Compositions Comprising Yeast Proteins from Cyberlindera

Cyberlindera jardinii yeasts were cultured to obtain a yeast cream. The yeast endogenous enzymes were inactivated by heating the yeast cream at 95° C. for 5 min. The resultant slurry was then cool down to 65° C. and the pH was adjusted to pH 9.0. These conditions were maintained for 30 min before the pH was reduced to 8.0 to perform an alkaline extraction. The resultant insoluble and soluble fractions were separated by centrifugation, and the insoluble fraction was collected prior to be subjected to a β-1,3-glucanase treatment (i.e., Denazyme GEL™, Nagase; 0.5% on a yeast dry matter basis) for 5 h at 55° C. and pH 5.5. The enzymatic reaction was stopped by heating the hydrolysate to 95° C. for at least 2 min (this step also served as a pasteurization step). The resultant insoluble and soluble fractions were separated by centrifugation, and the insoluble fraction was collected and then spray dried at 85° C. Protein content was determined via the Kjeldahl method of nitrogen analysis with 6.25 as coefficient. The yeast extract obtained comprised 83.97% proteins. While the invention has been described in connection with specific embodiments thereof, it will be understood that the scope of the claims should not be limited by the preferred embodiments set forth in the examples, but should be given the broadest interpretation consistent with the description as a whole.

### REFERENCES

[1] AOAC 980.13-1980; Title: Fructose, Glucose, Lactose, Maltose, and Sucrose in Milk Chocolate—Liquid Chromatography Method
[2] AOAC 989.05-1992; Title: Fat in Milk Modified Mojonnier Ether Extraction Method
[3] Brishti F. H. et al., (2017) Evaluation of the functional properties of mung bean protein isolate for development of textured vegetable protein. International Food Research Journal, 24 (4). pp. 1595-1605. ISSN 1985-4668; ESSN: 2231-7546
[4] Chandra S. et al., (2015) Evaluation of functional properties of composite flours and sensorial attributes of composite flour biscuits. J Food Sci Technol. 52 (6). pp. 3681-3688. doi: 10.1007/s13197-014-1427-2. Epub 2014 Jun. 10. PMID: 26028751; PMCID: PMC4444897
[5] Fish W. W. et al., (1991) A method for the quantitation of 5′-mononucleotides in foods and food ingredients. Journal of Agricultural and Food Chemistry. 39 (6), pp. 1098-1101. DOI: 10.1021/jf00006a019
[6] Onwulata C. I. et al., (2014) Rapid visco analysis of food protein pastes. Journal of Food Processing and Preservation. 38, pp. 2083-2089
[7] Yasumatsu K. et al., (1972) Whipping and Emulsifying Properties of Soybean Products, Agricultural and Biological Chemistry, 36 (5), pp. 719-727, DOI: 10.1080/00021369.1972.10860321

1. A method for obtaining a composition having yeast proteins, wherein the method comprises the following steps:
a) providing a yeast cream comprising yeasts;
b) inactivating the endogenous enzymes of the yeasts to provide an inactivated yeast cream;
c) subjecting the inactivated yeast cream to an enzymatic treatment to obtain an insoluble fraction comprising yeast protoplasts and a soluble fraction; wherein the enzymatic treatment comprises at least one polypeptide having a glucanase activity; and wherein the enzymatic treatment lacks ribonuclease activity;
d) separating the insoluble fraction from the soluble fraction; and
e) collecting the insoluble fraction, wherein the collected insoluble fraction, when dried, is the composition comprising the yeast proteins and has a protein content equal to or higher than 60% based on the total mass of the dried collected insoluble fraction.

2. The method of claim 1, wherein the inactivated yeast cream is obtained by exposing the yeast cream to a temperature of between 65 and 100° C., for a time of between 30 seconds and 5 hours.

3. The method of claim 1, wherein the enzymatic treatment is performed at a temperature of between 2° and 80° C., for a time of between 1 and 24 hours.

4. The method of claim 1, wherein the composition has a neutral taste.

5. The method of claim 1 further comprising drying the collected insoluble fraction of step e) to provide the composition.

6. The method of claim 5, wherein the dried insoluble has:
a lipid content lower than 20% based on the total mass of the dried collected insoluble fraction;
a nucleic acid content higher than 6% based on the total mass of the dried collected insoluble fraction;
a carbohydrate content lower than 25% based on the total mass of the dried collected insoluble fraction;
a mannan content lower than 6% based on the total mass of the dried collected insoluble fraction;
a glucan content lower than 10% based on the total mass of the dried collected insoluble fraction; and/or
a glucose content lower than 25% based on the total mass of the dried collected insoluble fraction.

7. The method of claim 1, further comprising, after b) and before c), submitting the inactivated yeast cream to an alkaline extraction step.

8. The method of claim 7, further drying the collected insoluble fraction of step e) to provide the composition.

9. The method of claim 8, wherein the dried insoluble fraction has:
a lipid content lower than 20% based on the total mass of the dried collected insoluble fraction;
a nucleic acid content lower than 3% based on the total mass of the dried collected insoluble fraction;
a carbohydrate content lower than 25% based on the total mass of the dried collected insoluble fraction;
a mannan content lower than 6% based on the total mass of the dried collected insoluble fraction;
a glucan content lower than 10% based on the total mass of the dried collected insoluble fraction; and/or
a glucose content lower than 25% based on the total mass of the dried collected insoluble fraction.

10. The method of claim 1, wherein the yeasts are from the genuses Saccharomyces, Komagataella, Pichia, Candida, Kluyveromyces, Yarrowia, Cyberlindera (Torula), Wickerhamomyces, or a combination thereof.

11. A composition comprising yeast proteins being derived from yeast protoplasts and having:
a protein content equal to or higher than 60% based on the total mass of the composition;
a lipid content lower than 20% based on the total mass of the composition;
a carbohydrate content lower than 25% based on the total mass of the composition;
a mannan content lower than 6% based on the total mass of the composition;
a glucan content lower than 10% based on the total mass of the composition; and/or
a glucose content lower than 25% based on the total mass of the composition.

12. The composition of claim 11 having a nucleic acid content higher than 6% based on the total mass of the composition.

13. The composition of claim 11 having a nucleic acid content lower than 3% based on the total mass of the composition.

14. The composition of claim 11 having a neutral taste.

15. The composition of claim 11 obtainable or obtained by the method of claim 1.

16. An edible product comprising the composition of claim 11 and at least one further ingredient, wherein the composition provides at least 1% w/w, based on the total mass of edible product.

17. The edible product of claim 16 being a beverage, a shake, a bar, a meat product or a baked product.

18. The edible product of claim 16, wherein the edible product is a human and/or animal nutrition.

19. The edible product of claim 18, wherein the edible product provides as an alternative to animal or plant/leguminous proteins-based edible products, or is combined with animal or plant/leguminous proteins-based edible products.

20. The edible product of claim 16, wherein the edible product is a food/feed supplement or food/feed additive.

21. (canceled)
